(19) United States
(12) Patent Application Publication
(10) Pub. No.: US 2025/0075224 A1
(43) Pub. Date: Mar. 6, 2025

(54) PLANTS WITH IMPROVED DIGESTIBILITY AND MARKER HAPLOTYPES

(71) Applicant: KWS SAAT SE & Co. KGaA, Einbeck (DE)

(72) Inventors: Monika KLOIBER-MAITZ, Einbeck (DE); Therese BOLDUAN, Eibeck (DE); Milena OUZUNOVA, Göttingen (DE); Nina MEYER, Einbeck (DE); Carolina LOPEZ-DURAN, Einbeck (DE)

(73) Assignee: KWS SAAT SE & Co. KGaA, Einbeck (DE)

(21) Appl. No.: 18/952,425

(22) Filed: Nov. 19, 2024

Related U.S. Application Data
(63) Continuation of application No. 17/049,368, filed on Oct. 21, 2020, now Pat. No. 12,180,490.

(30) Foreign Application Priority Data
Apr. 24, 2018 (EP) 18169122.1

(51) Int. Cl. C12N 15/82; A01H 1/04; C12N 9/02; C12Q 1/6895

(52) U.S. Cl. CPC C12N 15/8242; A01H 1/045; C12N 9/0071; C12Q 1/6895; C12Y 114/13088; C12Q 2600/13; C12Q 2600/156

(57) ABSTRACT

The present invention relates to plants, such as maize, sorghum or sugar cane, having improved digestibility, in particular improved stover digestibility. The present invention relates to a QTL allele associated with improved digestibility and specific marker alleles associated with the QTL allele. The present invention further relates to such plants, wherein the F35H gene is mutated or wherein F35H expression is altered. The invention also relates to methods for identifying plants having improved digestibility and methods for obtaining such plants.

### CROSS-REFERENCE TO RELATED APPLICATIONS

This application is a continuation of U.S. application Ser. No. 17/049,368, filed Oct. 21, 2020, which is a U.S. National Phase of International Patent Application No. PCT/EP2019/060411, filed on Apr. 24, 2019, which claims priority to EP application Ser. No. 18/169,122.1, filed Apr. 24, 2018. The entire contents of these applications are incorporated herein by reference in their entirety.

### SEQUENCE LISTING

The instant application contains a Sequence Listing which has been submitted electronically in XML format and is hereby incorporated by reference in its entirety. Said XML copy, created on Nov. 8, 2024, is named 245761_000247_SL.xml and is 125,936 bytes in size.

### FIELD OF THE INVENTION

The invention relates to quantitative trait loci (QTL) and associated markers involved in and/or associated with improved digestibility of plants and plant parts, such as maize. The invention further relates to uses of such QTL or markers for identification and/or selection purposes, as well as transgenic or non-transgenic plants.

### BACKGROUND OF THE INVENTION

Maize (Zea mays L.) is the most important annual forage crop in the world. More than 3 million hectares of maize are ensiled each year, mainly in Northern Europe. Due to high energy content and feed conversion efficiency, the forage maize is an important food crop for dairy and beef cattle, and is affecting significantly the milk and meat production. There is a wide genetic variation in forage characteristics for both the entire maize plant and stover (Geiger et al. 1992; Barrière et al. 2003).

Therefore, improving digestibility is a major goal for forage maize breeding programs. The energy supplied by forage to a ruminant or herbivore animal diet is related to forage ingestibility and digestibility. The digestibility of any forage constituent (dry matter, organic matter, or cell wall) is measured as percentage of silage absorbed in the animal digestive tract (Barrière et al. 2003). The overall digestibility of forage maize is affected by the highly digestible grain and stover fraction. Stover composition and digestibility limits forage maize quality. Major stover fractions are hemicelluloses, cellulose, and lignins. Modern forage maize cultivars combine high dry matter yield with high stover digestibility.

It is too costly to perform digestibility measurements with animals, especially when conducting large scale evaluation of germplasm in plant breeding programs. Biological and chemical methods have been developed to assay the digestibility of maize and other forage crops (Van Soest et al. 1963). Neutral detergent fiber (NDF), the residual after removing cell soluble content, is an important plant cell wall and cellulose indicator. In vitro NDF digestibility (IVNDFD) of forages is an estimate of cell wall digestibility assuming that the non-NDF part of plant material was completely digestible (Méchin et al. 2000). Additionally, the use of NIRS has been reported to measure digestibility traits accurately in many forage crops including maize (Lubberstedt et al. 1997a, b; Zimmer et al. 1990).

Lübberstedt et al. (1997a, b) first published QTL related to forage maize agronomic and quality traits, and QTL for whole plant digestibility. Exploiting available genetic variation for stover digestibility by marker-assisted selection (MAS) seems to be a promising way to improve forage digestibility. Besides genetic variance, environmental variation might be the reason for those inconsistent traits. QTL analyses of forage traits in four different maize populations revealed only few QTL showing epistatic interactions or interactions with the environment (Lübberstedt et al. 1998). Seven QTL for DNDF were detected by using 242 RILs derived from the cross F838×F286 which were evaluated in per se value experiments in six environments, and found two major QTL (Barrière et al. 2010). Additional QTL analyses were conducted by using RIL progeny derived from a cross between an old dent and modern lodent lines, and new QTL in bins 2.06 and 5.04 for ADL/NDF and DNDF were first reported (Barrière et al. 2012).

It is therefore an objective of the present invention to address one or more of the shortcomings of the prior art. There is a persistent need for improving digestibility of fodder crops, as well as the identification of plants, including particular plant parts or derivatives having increased digestibility. In particular, it is an aim of the present invention to provide new major QTL for digestibility and the causative gene(s) and the provision of markers which allow the economical use of these QTL in maize development and breeding.

### SUMMARY OF THE INVENTION

The present invention is based on the identification of a major QTL for plant digestibility as well as the identification of a F35H gene linked to and responsible for the QTL for plant digestibility and description of a unique marker haplotype for improved digestibility. Molecular markers have been identified which are associated with plant digestibility, and marker alleles associated with improved digestibility are described. One of the described marker alleles is a mutated F35H gene.

The invention in particular relates to methods for detecting the identified QTL allele associated with improved digestibility, as well as detection of any of the described marker alleles. The invention further relates to the described marker alleles and polynucleic acids useful for detection of the marker alleles, such as primers and probes, and kits comprising such. The invention further relates to methods for improving plant digestibility, in particular by naturally or artificially introducing in plants and/or selecting plants comprising, the marker alleles described herein, such as in particular inducing F35H mutations, preferably mutations altering F35H expression or F35H enzymatic activity, e.g. reducing or eliminating F35H expression or F35H activity or otherwise reducing

F35H expression or F35H activity, or increasing F35H activity. The invention further relates to plants having improved digestibility, as well as plant parts, in particular stover, having improved digestibility.

The present invention is in particular captured by any one or any combination of one or more of the below numbered items to [25], with any other item and/or embodiments.

[01] A method for identifying a plant or plant part having improved digestibility or for selecting a plant or plant part having improved digestibility comprising
(i) optionally, isolating genetic material, preferably genetic material, from at least one cell of the plant or plant part;
(ii) a) screening for the presence of a QTL allele, such as a QTL allele associated with improved digestibility, said QTL allele comprising a nucleotide sequence of a gene encoding a cytochrome P450 flavonoid 3′,5′-hydroxylase (F35H), having a mutation, preferably a mutation leading to altered expression of the mRNA of the gene and/or the F35H protein, or a mutation leading to an F35H protein having altered enzymatic activity upon translation, more preferably a mutation leading to reduced or absent expression of the mRNA of the gene and/or the F35H protein (such as a knock-down or knock-out mutation), or a mutation leading to a non-functional F35H protein (e.g., truncated F35H protein) or an F35H protein having reduced enzymatic activity upon translation, or an F35H protein having increased enzymatic activity upon translation, or
(ii) b) screening for altered expression of the mRNA of a gene encoding a cytochrome P450 flavonoid 3′,5′-hydroxylase (F35H) and/or the F35H protein or for an F35H protein having altered enzymatic activity, preferably for reduced or absent expression of the mRNA of a gene encoding a cytochrome P450 flavonoid 3′,5′-hydroxylase (F35H) and/or the F35H protein or for a non-functional F35H protein (e.g., truncated F35H protein) or an F35H protein having reduced enzymatic activity, or for an F35H protein having increased enzymatic activity or
(ii) c) screening for the presence of a mutation leading to altered expression of the mRNA of a gene encoding a cytochrome P450 flavonoid 3′,5′-hydroxylase (F35H) and/or the F35H protein, or a mutation, preferably a mutation leading to an F35H protein having altered enzymatic activity upon translation, preferably leading to reduced or absent expression of the mRNA of a gene encoding a cytochrome P450 flavonoid 3′,5′-hydroxylase (F35H) and/or the F35H protein (such as a knock-down or knock-out mutation), or a mutation, preferably a mutation leading to a non-functional F35H protein (e.g., truncated F35H protein) or an F35H protein having reduced enzymatic activity upon translation or an F35H protein having increased enzymatic activity upon translation;
(iii) optionally selecting the plant or plant part in which the QTL or the mutation is present or in which the F35H mRNA and/or protein expression is altered or the enzymatic F35H activity is altered, preferably the F35H mRNA and/or protein expression is reduced or eliminated or the enzymatic F35H activity is reduced or increased.

[02] The method according to item [01], preferably wherein said plant is a maize plant, the method comprising screening for the presence of the molecular marker allele of ma61134xxx and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

[03] A plant or plant part comprising a QTL allele, such as a QTL allele associated with improved digestibility, said QTL allele comprising a nucleotide sequence of a gene encoding a cytochrome P450 flavonoid 3′,5′-hydroxylase (F35H), having a mutation preferably a mutation leading to altered expression of the mRNA of the gene and/or the F35H protein, or a mutation leading to an F35H protein having altered enzymatic activity upon translation, more preferably a mutation leading to reduced or absent expression of the mRNA of the gene and/or the F35H protein, or a mutation leading to a non-functional F35H protein (e.g., truncated F35H protein) or an F35H protein having reduced enzymatic activity upon translation, or an F35H protein having increased enzymatic activity upon translation; or a plant or plant part comprising a nucleotide sequence of a gene encoding a cytochrome P450 flavonoid 3′,5′-hydroxylase (F35H), having a mutation, preferably a mutation leading to altered expression of the mRNA of the gene and/or the F35H protein, or a mutation leading to an F35H protein having altered enzymatic activity upon translation, more preferably a mutation leading to reduced or absent expression of the mRNA of the gene and/or the F35H protein, or a mutation leading to a non-functional F35H protein (e.g., truncated F35H protein) or an F35H protein having reduced enzymatic activity upon translation, or an F35H protein having increased enzymatic activity upon translation; or a plant or plant part comprising a nucleotide sequence of a gene encoding a cytochrome P450 flavonoid 3′,5′-hydroxylase (F35H) having reduced or absent expression of the mRNA of the gene and/or the F35H protein or having reduced enzymatic activity, preferably having reduced enzymatic activity or having increased enzymatic activity; or a plant or plant part comprising an RNAi molecule, such as dsRNA, siRNA, shRNA, or miRNA, directed against, targeting, or hybridizing with a nucleotide sequence encoding an F35H protein, or comprising a polynucleotide sequence encoding (and expressing or being capable of expressing) an RNAi molecule directed against, targeting, or hybridizing with a nucleotide sequence encoding an F35H protein; or a plant or plant part comprising an RNA-specific CRISPR/Cas system, such as a CRISPR/Cas13a system, directed against or targeting a nucleotide sequence encoding a cytochrome P450 flavonoid 3′,5′-hydroxylase (F35H) or one or more polynucleotide sequence(s) encoding (and expressing or being capable of expressing) said RNA-specific CRISPR/Cas system.

[04] The plant or plant part according to item [03], preferably wherein said plant is a maize plant, comprising the marker allele of ma61134xxx.

[05] The plant or plant part according to item or [04], wherein said plant comprising said QTL allele, said marker allele, and/or said nucleotide sequence of the gene encoding the cytochrome P450 flavonoid 3′,5′-hydroxylase (F35H) having the mutation as introgression.

[06] The plant or plant part according to item or [04], wherein said plant comprising said QTL allele, said marker allele, said nucleotide sequence of the gene encoding the cytochrome P450 flavonoid 3′,5′-hydroxylase (F35H) having the mutation, said RNAi molecule or said polynucleotide sequence encoding (and expressing or being capable of expressing) the RNAi molecule, said RNA-specific CRISPR/Cas system and/or said one or more polynucleotide sequence(s) encoding (and expressing or being capable of expressing) the RNA-specific CRISPR/Cas system as transgene or as (gene-) edited endogene.

[07] A method for improving digestibility of a plant or plant part, comprising introducing or introgressing into the genome of a plant or plant part
(a) a nucleotide sequence of a gene encoding a cytochrome P450 flavonoid 3′,5′-hydroxylase (F35H), having a mutation, preferably a mutation leading to altered expression of the mRNA of the gene and/or the F35H protein, or a mutation leading to an F35H protein having altered enzymatic activity upon translation, more preferably a mutation leading to reduced or absent expression of the mRNA of the gene and/or the F35H protein (such as a knock-down or knock-out mutation), or a mutation leading to a non-functional F35H protein (e.g., truncated F35H protein) or an F35H protein having reduced enzymatic activity upon translation, or an F35H protein having increased enzymatic activity upon translation, or
(b) a QTL allele, such as a QTL allele associated with improved digestibility, and comprising a nucleotide sequence of a gene encoding a cytochrome P450 flavonoid 3′,5′-hydroxylase (F35H), having a mutation, preferably a mutation leading to altered expression of the mRNA of the gene and/or the F35H protein, or a mutation leading to an F35H protein having altered enzymatic activity upon translation, more preferably a mutation leading to reduced or absent expression of the mRNA of the gene and/or the F35H protein (such as a knock-down or knock-out mutation), or a mutation leading to a non-functional F35H protein (e.g., truncated F35H protein) or an F35H protein having reduced enzymatic activity upon translation, or an F35H protein having increased enzymatic activity upon translation.

[08] A method for improving digestibility of a plant or plant part, comprising altering the expression of the mRNA of a gene encoding a cytochrome P450 flavonoid 3′,5′-hydroxylase (F35H) and/or the F35H protein, altering the enzymatic activity of a cytochrome P450 flavonoid 3′,5′-hydroxylase (F35H), preferably reducing, eliminating or inhibiting expression of the mRNA of a gene encoding a cytochrome P450 flavonoid 3′,5′-hydroxylase (F35H) and/or the F35H protein, reducing the enzymatic activity of a cytochrome P450 flavonoid 3′,5′-hydroxylase (F35H), inhibiting the F35H protein, or increasing the enzymatic activity of a cytochrome P450 flavonoid 3′,5′-hydroxylase (F35H).

[09] The method according to item [08], comprising
(a) introducing into a nucleotide sequence of an endogenous gene encoding the cytochrome P450 flavonoid 3′,5′-hydroxylase (F35H) (in the genome of the plant or the plant part) a mutation, preferably a mutation leading to altered expression of the mRNA of the gene and/or the F35H protein, or a mutation leading to an F35H protein having altered enzymatic activity upon translation, more preferably a mutation leading to reduced or absent expression of the mRNA of the gene and/or the F35H protein (such as a knock-down or knock-out mutation), or a mutation leading to a non-functional F35H protein (e.g., truncated F35H protein) or an F35H protein having reduced enzymatic activity upon translation, or an F35H protein having increased enzymatic activity upon translation, or
(b) introducing into the plant or the plant part an RNAi molecule, such as dsRNA, siRNA, shRNA, or miRNA, directed against, targeting, or hybridizing with a nucleotide sequence encoding the F35H protein, or a polynucleotide sequence encoding (and expressing or being capable of expressing) an RNAi molecule directed against, targeting, or hybridizing with a nucleotide sequence encoding the F35H protein, or
(c) introducing into the plant or the plant part an RNA-specific CRISPR/Cas system, such as a CRISPR/Cas13a system, directed against or targeting a nucleotide sequence encoding the F35H protein, or one or more polynucleotide sequence(s) encoding (and expressing or being capable of expressing) said RNA-specific CRISPR/Cas system, or
(d) introducing into the plant or the plant part a chemical compound or an antibody altering (or being capable to alter) the enzymatic activity of the F35H protein upon interaction with said F35H, preferably reducing (or being capable to reduce) the enzymatic activity of the F35H protein or inhibiting (or being capable to inhibit) the enzymatic activity of the F35H protein or increasing (or being capable to increase) the enzymatic activity of the F35H protein upon interaction with said F35H.

[10] A method for producing a plant or plant part having improved digestibility, comprising
(a) introducing or introgressing into the genome of a plant or plant part a nucleotide sequence of a gene encoding a cytochrome P450 flavonoid 3′,5′-hydroxylase (F35H), having a mutation, preferably a mutation leading to altered expression of the mRNA of the gene and/or the F35H protein, or a mutation leading to an F35H protein having altered enzymatic activity upon translation, more preferably a mutation leading to reduced or absent expression of the mRNA of the gene and/or the F35H protein (such as a knock-down or knock-out mutation), or a mutation leading to a non-functional F35H protein (e.g., truncated F35H protein) or an F35H protein having reduced enzymatic activity upon translation, or an F35H protein having increased enzymatic activity upon translation, or
(b) introducing or introgressing into the genome of a plant or plant part a QTL allele, such as a QTL allele associated with improved digestibility, and comprising a nucleotide sequence of a gene encoding a cytochrome P450 flavonoid 3′,5′-hydroxylase (F35H), having a mutation, preferably a mutation leading to altered expression of the mRNA of the gene and/or the F35H protein, or a mutation leading to an F35H protein having altered enzymatic activity upon translation, more preferably a mutation leading to reduced or absent expression of the mRNA of the gene and/or the F35H protein (such as a knock-down or knock-out mutation), or a mutation leading to a non-functional F35H protein (e.g., truncated F35H protein) or an F35H protein having reduced enzymatic activity upon translation, or an F35H protein having increased enzymatic activity upon translation.
(c) introducing into a nucleotide sequence of an endogenous gene encoding the cytochrome P450 flavonoid 3′,5′-hydroxylase (F35H) (in the genome of the plant or a plant part) a mutation, preferably a mutation leading to altered expression of the mRNA of the gene and/or the F35H protein, or a mutation leading to an F35H protein having altered enzymatic activity upon translation, more preferably a mutation leading to reduced or absent expression of the mRNA of the gene and/or the F35H protein (such as a knock-down or knock-out mutation), or a mutation leading to a non-functional F35H protein or an F35H protein having reduced enzymatic activity upon translation, or an F35H protein having increased enzymatic activity upon translation, or
(d) introducing into the plant or the plant part an RNAi molecule, such as dsRNA, siRNA, shRNA, or miRNA, directed against, targeting, or hybridizing with a nucleotide sequence encoding the F35H protein, or a polynucleotide sequence encoding (and expressing or being capable of expressing) an RNAi molecule directed against, targeting, or hybridizing with a nucleotide sequence encoding the F35H protein, or
(e) introducing into the plant or the plant part an RNA-specific CRISPR/Cas system, such as a CRISPR/Cas13a system, directed against or targeting a nucleotide sequence encoding the F35H protein, or one or more polynucleotide sequence(s) encoding (and expressing or being capable of expressing) said RNA-specific CRISPR/Cas system, or
(f) introducing into the plant or the plant part a chemical compound or an antibody altering (or being capable to alter) the enzymatic activity of the F35H protein upon interaction with said F35H, preferably reducing (or being capable to reduce) the enzymatic activity of the F35H protein or inhibiting (or being capable to inhibit) the F35H protein or increasing (or being capable to increase) the enzymatic activity of the F35H protein upon interaction with said F35H; and
(g) optionally, regenerating a plant from the plant part of any of (a) to (e).

[11] A plant or plant part produced by the method according to item [10].

[12] A progeny of the plant according to any one of item to or [11].

[13] The method, plant, or plant part according to any of the preceding items, wherein said plant is a maize plant, the QTL is located on chromosome 9 and comprises and/or is flanked by marker alleles ma61070s01 and ma30168s02, preferably by marker alleles ma50827s01 and ma16983s02, more preferably by marker alleles ma17117s01 and ma61125s01.

[14] The method, plant, or plant part according to any of the preceding items, wherein
ma61134xxx is an insertion of one or more nucleotides between position 134254381 and 134254382 of chromosome 9 referenced to line PH207, preferably an insertion as set forth in SEQ ID NO: 12; and/or
ma61070s01 is a single nucleotide polymorphism (SNP) at position 121588825 of chromosome 9 referenced to line PH207, wherein said nucleotide is A or T, preferably a single nucleotide polymorphism (SNP) as set forth in SEQ ID NO: 13; and/or
ma30168s02 is a single nucleotide polymorphism (SNP) at position 139452428 of chromosome 9 referenced to line PH207, wherein said nucleotide is A or G, preferably a single nucleotide polymorphism (SNP) as set forth in SEQ ID NO: 14; and/or
ma50827s01 is a single nucleotide polymorphism (SNP) at position 127454426 of chromosome 9 referenced to line PH207, wherein said nucleotide is A or G, preferably a single nucleotide polymorphism (SNP) as set forth in SEQ ID NO: 15; and/or
ma16983s02 is a single nucleotide polymorphism (SNP) at position 137363784 of chromosome 9 referenced to line PH207, wherein said nucleotide is A or G, preferably a single nucleotide polymorphism (SNP) as set forth in SEQ ID NO: 16; and/or
ma17117s01 is a single nucleotide polymorphism (SNP) at position 132038900 of chromosome 9 referenced to line PH207, wherein said nucleotide is A or G, preferably a single nucleotide polymorphism (SNP) as set forth in SEQ ID NO: 17; and/or
ma61125s01 is a single nucleotide polymorphism (SNP) at position 135947973 of chromosome 9 referenced to line PH207, wherein said nucleotide is A or G, preferably a single nucleotide polymorphism (SNP) as set forth in SEQ ID NO: 18.

[15] The method, plant, or plant part according to any of the preceding items, wherein the nucleotide sequence of the unmutated F35H gene comprises a sequence selected from the group consisting of:
(i) a nucleotide sequence of SEQ ID NO: 1, 4, or 7;
(ii) a nucleotide sequence having the cDNA of SEQ ID NO: 2, 5, or 8;
(iii) a nucleotide sequence encoding for an amino acid sequence of SEQ ID NO: 3, 6, or 9;
(iv) a nucleotide sequence having at least 60% identity to the sequence of SEQ ID NO: 1, 2, 4, 5, 7, or 8;
(v) a nucleotide sequence encoding for a polypeptide having at least 60% identity to the sequence of SEQ ID NO: 3, 6, or 9;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s) of the amino acid sequence encoded by the nucleotide sequence of (i) to (vi).

[16] The method, plant, or plant part according to any of the preceding items, wherein the mutation is a frameshift mutation or a non-sense-mutation, results in an altered expression of the nucleotide sequence or an altered enzymatic activity of the encoded protein, preferably in a reduced or absent expression of the nucleotide sequence or a reduced enzymatic activity of the encoded protein or an increased enzymatic activity of the encoded protein, results in an altered protein sequence encoded by the nucleotide sequence, or is an insertion, deletion or substitution of at least one nucleotide in a coding region, in a splicing signal or in a regulatory element of said nucleotide sequence.

[17] The method, plant, or plant part according to any of the preceding items, wherein the mutation is an insertion, preferably in an exon, preferably an insertion in the first exon, of one or more nucleotides, preferably a frame shift insertion, more preferably the insertion is 187 nucleotides or about 187 nucleotides and/or the insertion is between position 97 and 98 of the F35H gene represented by the nucleotide sequence of SEQ ID NO: 1. In a particular preferred embodiment the mutated F35H comprises the nucleotide sequence of SEQ ID NO: 11. Alternatively, the mutation is a substitution, preferably a substitution of at least one nucleic acid resulting in an exchange of at least one amino acid or resulting in the change of an amino acid coding codon into a stop codon. In preferred embodiments, the mutated F35H comprises the nucleotide sequence encoding one of the amino acid sequences selected from the group consisting of SEQ ID NO: 20, 22, 24, 26, 28, 30, 32, 34, 36, 38 or 40. Such nucleotide sequence may be selected from the group consisting of SEQ ID NO: 19, 21, 23, 25, 27, 29, 31, 33, 35, 37 or 39.

[18] The method, plant or plant part according to any of the preceding items, wherein said plant part is not propagation material.

[19] The method, plant, or plant part according to any of the preceding items, wherein said plant part is stover.

[20] The method, plant, or plant part according to any of the preceding items, wherein the plant is or the plant part is from maize, sorghum or sugar cane.

[21] A polynucleic acid, such as an allele specific polynucleic acid (molecular marker), specifically hybridising with any of the sequences of SEQ ID NO: 10, 12, 13, 14, 15, 16, 17 or 18, or the complement or the reverse complement thereof.

[22] Use of the polynucleic acid according to item or polynucleic acid, such as an allele specific polynucleic acid (molecular marker), for identification of a plant or plant part having improved digestibility or for selection of a plant or plant part having improved digestibility according to any one of item to [06], preferably in the method according to item or [02], wherein preferably the polynucleic acid is suitable for hybridization as forward primer and reverse primer to a locus in a chromosomal interval which co-segregates with the improved digestibility, wherein the chromosomal interval is on chromosome 9 and flanked by marker alleles ma61070s01 and ma30168s02, preferably by marker alleles ma50827s01 and ma16983s02, more preferably by marker alleles ma17117s01 and ma61125s01.

[23] A method for producing an ensilaged plant material or animal feed having improved digestibility, comprising
(a) growing the plant according to any one of item to or [11],
(b) harvesting the plant or a part thereof,
(c) optionally, chopping and/or crushing the plant or a part thereof, and
(d) ensiling the plant or a part thereof of (b) or (c), optionally by adding a stimulant like a bacterial inoculant, a sugar, and an enzyme.

[24] An ensilaged plant material or animal feed produced by the method of item [23].

[25] A method for producing biogas or bioethanol, comprising the following steps:
(a) providing the plant according to any one of item to or or the ensilaged plant material according to item [24], and
(b) producing biogas or bioethanol from the plant or the ensilaged plant material.

### BRIEF DESCRIPTION OF THE FIGURES

FIG. 1: DNDF (Digestible Neutral Detergent Fiber) effects of QTL on chomosome 9 of maize (Zea mays). Identification of a strong QTL for digestibility on chromosome 9. Percentage of DNDF of maize stover is indicated.

FIG. 2: Positions of marker loci for silage QTL. Markers have been found by SeqCapture on the basis of AGPv02 and WGS (whole genome sequencing) of QTL line and comparison to PH207 reference.

FIG. 3: Fine-mapped genetic region of silage QTL by molecular markers in different recombinant genotypes.

FIG. 4: Nucleotide sequence alignment of an F35H reference gene and a mutated F35H gene according to an embodiment of the invention.

FIG. 5: Functional validation by RNAseq experiment. Arrow shows that the insertion is expressed and causes a frameshift.

FIG. 6: Greenhouse trial for fast validation of Mutants for the F35H gene: Digestibility results of mutant and wildtype plants for identified QTL. On average PH207m023a mutant and wildtype showed a difference of 7.3 DNDF units.

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Preferred items (features) and embodiments of this invention are set herein below. Each items and embodiments of the invention so defined may be combined with any other item and/or embodiments unless clearly indicated to the contrary. In particular, any feature indicated as being preferred or advantageous may be combined with any other feature or features or items indicated as being preferred or advantageous.

As used herein, “maize” refers to a plant of the species Zea mays, preferably Zea mays ssp mays.

As used herein, “sorghum” refers to a plant of the genus Sorghum, and includes without limitation Sorghum bicolor, Sorghum sudanense, Sorghum bicolor×Sorghum sudanense, Sorghum×almum (Sorghum bicolor×Sorghum halepense), Sorghum arundinaceum, Sorghum×drummondii, Sorghum halepense and/or Sorghum propinquum.

As used herein, “sugar cane” refers to a plant of the species Saccharum officinarum.

The term “plant” includes whole plants, including descendants or progeny thereof. The term “plant part” includes any part or derivative of the plant, including particular plant tissues or structures, plant cells, plant protoplast, plant cell or tissue culture from which plants can be regenerated, plant calli, plant clumps and plant cells that are intact in plants or parts of plants, such as seeds, kernels, cobs, flowers, cotyledons, leaves, stems, buds, roots, root tips, stover, and the like. Plant parts may include processed plant parts or derivatives, including flower, oils, extracts etc. In certain embodiments, the plant part or derivative as referred to herein is stover.

Stover as used herein has its ordinary meaning known in the art. By means of further guidance, and without limitation, stover may comprise, consist of, or consist essentially of the leaves and stalks of field crops, such as maize or sorghum that are commonly left in a field after harvesting the grain, or as sugar cane. Stover may also include cobs (e.g. the central core of an ear of maize, without the kernels). Stover may also exclude cobs. Stover may also include husks or hulls (e.g. the leafy outer covering of an ear of maize). Stover may also exclude husks or hulls. Stover is similar to straw, the residue left after any cereal grain or grass has been harvested at maturity for its seed. It can be directly grazed by cattle or dried for use as fodder. (Maize) stover can be used as feed, whether grazed as forage, chopped as silage to be used later for fodder, or collected for direct (non-ensilaged) fodder use. Maize forage is usually ensiled in cooler regions, but it can be harvested year-round in the tropics and fed as green forage to the animals. In the silage use case, it is usual for the entire plant (grain and stover together) to be chopped into pieces which are then crushed between rollers while harvesting. In addition to the stalks, leaves, husks, and cobs remaining in the field, kernels of grain may also be left over from harvest. These left over kernels, along with the corn stover, serve as an additional feed source for grazing cattle.

In certain embodiments, the plant part or derivative comprises, consists of, or consists essentially of one or more, preferably all of stalks, leaves, and cobs. In certain embodiments, the plant part or derivative is leaves. In certain embodiments, the plant part or derivative is stalks. In certain embodiments, the plant part or derivative is cobs.

In certain embodiments, the plant part or derivative comprises, consists of, or consists essentially of one or more, preferably all of stalks and leaves. In certain embodiments, the plant part or derivative comprises, consists of, or consists essentially of one or more, preferably all of stalks, and cobs. In certain embodiments, the plant part or derivative comprises, consists of, or consists essentially of one or more, preferably all of leaves and cobs. In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, ensilaging etc.

As used herein, “digestibility” refers to and is measured as percentage of product (such as a maize, sorghum or sugar cane plant or plant part or derivative, including for instance dry matter, organic matter, or cell wall of the product) absorbed in the animal digestive tract (Barrière et al. 2003). Biological and chemical methods have been developed to assay the digestibility of maize and other forage crops (Van Soest et al. 1963). Neutral detergent fiber (NDF), the residual after removing cell soluble content, is an important plant cell wall and cellulose indicator. In vitro NDF digestibility (IVNDFD) of forages is an estimate of cell wall digestibility assuming that the non-NDF part of plant material was completely digestible (Mechin et al. 2000). Additionally, the use of NIRS has been reported to measure digestibility traits accurately in many forage crops including maize (Lübberstedt et al. 1997a, b; Zimmer et al. 1990). In certain embodiments, the animal is a mammal. In certain embodiments, the animal is a ruminant. In certain embodiments, the animal is a herbivore. In certain embodiments, the animal is a herbivorous mammal.

Improved digestibility as referred to herein, relates to increased digestibility of a plant or plant part or derivative having a characteristic according to the invention, such as a mutation, marker, SNP, or QTL as described herein elsewhere, compared to a plant or plant part or derivative not having such characteristic. In certain embodiments, an improved or increased digestibility refers to an increase in mean DNDF by at least 1%, such as at least 2%, at least 3%, at least 4%, at least 5%, at least 6%, at least 7%, at least 8%, at least 9%, or at least 10%, preferably at least 2%, more preferably at least 3%, such as at least 4%.

The term “locus” (loci plural) means a specific place or places or a site on a chromosome where for example a QTL, a gene or genetic marker is found. As used herein, the term “quantitative trait locus” or “QTL” has its ordinary meaning known in the art. By means of further guidance, and without limitation, a QTL may refer to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more monomorphic and/or polymorphic markers. A haplotype can be defined by the unique fingerprint of alleles at each marker within the specified window. A QTL may encode for one or more alleles that affect the expressivity of a continuously distributed (quantitative) phenotype. In certain embodiments, the QTL as described herein may be homozygous. In certain embodiments, the QTL as described herein may be heterozygous.

As used herein, the term “allele” or “alleles” refers to one or more alternative forms, i.e. different nucleotide sequences, of a locus.

As used herein, the term “mutant alleles” or “mutation” of alleles include alleles having one or more mutations, such as insertions, deletions, stop codons, base changes (e.g., transitions or transversions), or alterations in splice junctions/splicing signal sites, which may or may not give rise to altered gene products. Modifications in alleles may arise in coding or non-coding regions (e.g. promoter regions, exons, introns or splice junctions).

As used herein, the terms “introgression”, “introgressed” and “introgressing” refer to both a natural and artificial process whereby chromosomal fragments or genes of one species, variety or cultivar are moved into the genome of another species, variety or cultivar, by crossing those species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three quarter or half of a chromosome, but is preferably smaller, such as about 50 Mb or less, such as about 30 Mb or less, about 20 Mb or less, about 25 Mb or less, about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

A genetic element, a locus, an introgression fragment, an QTL, or a gene or allele conferring a trait (such as improved digestibility) is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, QTL, gene or allele. The terms are used interchangeably and the genetic element, locus, introgression fragment, QTL, gene or allele can thus be transferred into any other genetic background lacking the trait. Not only pants comprising the genetic element, locus, introgression fragment, QTL, gene or allele can be used, but also progeny/descendants from such plants which have been selected to retain the genetic element, locus, introgression fragment, QTL, gene or allele, can be used and are encompassed herein. Whether a plant (or genomic DNA, cell or tissue of a plant) comprises the same genetic element, locus, introgression fragment, QTL, gene or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic or gene-edited plants may also be encompassed.

As used herein the terms “transformation” and “transgenic modification” are all used herein as synonyms for the transfer of isolated and cloned nucleic acid molecule into the DNA, usually the chromosomal DNA or genome, of another organism/species or of the same organism/species but at a location which differs from the location at which the nucleic acid molecule naturally is located in the chromosomal DNA or genome.

“Introducing” in the meaning of the present invention includes stable or transient integration by means of transformation including Agrobacterium-mediated transformation, transfection, microinjection, biolistic bombardment, insertion using gene editing technology like CRISPR systems (e.g. CRISPR/Cas, in particular CRISPR/Cas9 or CRISPR/Cpf1), CRISPR/CasX, or CRISPR/CasY), TALENs, zinc finger nucleases or meganucleases, homologous recombination optionally by means of one of the below mentioned gene editing technology including preferably a repair template, modification of endogenous gene using random or targeted mutagenesis like TILLING or above mentioned gene editing technology, etc.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene” or “exogene” refers to a nucleic acid molecule or a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”. “Endogene” refers to a nucleic acid molecule or a genetic locus that naturally occurs in the genome of a plant. “Gene editing” or “genome editing” refers to genetic engineering in which DNA or RNA is inserted, deleted, modified or replaced in the genome of an organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas) system. These nucleases create site-specific double-strand breaks (DSBs) at desired locations in the genome. The induced double-strand breaks are repaired through nonhomologous end-joining (NHEJ) or homologous recombination (HR) or homology directed repair (HDR), resulting in targeted mutations or nucleic acid modifications. The use of designer nucleases, optionally together with a repair template/recombination template, is particularly suitable for generating gene knockouts or knockdowns. In certain embodiments, designer nucleases are developed which specifically induce a mutation in the F35H gene, as described herein elsewhere, such as to generate a mutated F35H or a knockout of the F35H gene. In certain embodiments, designer nucleases, in particular RNA-specific CRISPR/Cas systems are developed which specifically target the F35H mRNA, such as to cleave the F35H mRNA and generate a knockdown of the F35H gene/mRNA/protein. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As uses herein, “hybridization” or “hybridizing” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PGR, or the cleavage of a polynucleotide by an enzyme. A sequence capable of hybridizing with a given sequence is referred to as the “complement” of the given sequence.

Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance, a genetic element may be replaced by gene editing in which a recombination template is provided. The DNA may be cut upstream and/or downstream of a sequence which needs to be replaced. As such, the sequence to be replaced is excised from the DNA. Through HDR, the excised sequence is then replaced by the template. In certain embodiments, the QTL allele of the invention as described herein may be provided on/as a template. By designing the system such that double strand breaks are introduced upstream and/or downstream of the corresponding region in the genome of a plant not comprising the QTL allele, this region is excised and can be replaced with the template comprising the QTL allele of the invention. In this way, introduction of the QTL allele of the invention in a plant need not involve multiple backcrossing, in particular in a plant of specific genetic background. Similarly, the mutated F35H of the invention may be provided on/as a template. More advantageously however, the mutated F35H of the invention may be generated without the use of a recombination template, but solely through the endonuclease action leading to a double strand DNA break which is repaired by NHEJ, resulting in the generation of indels.

In certain embodiments, the nucleic acid modification or mutation is effected by a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle EL. Christian M. Wang L. Zhang Y. Schmidt C, et al. Efficient design and assembly of custom TALEN and other TAL effector-based constructs for DNA targeting. Nucleic Acids Res. 2011;39: e82; Zhang F. Cong L. Lodato S. Kosuri S. Church GM. Arlotta P Efficient construction of sequence-specific TAL effectors for modulating mammalian transcription. Nat Biotechnol. 2011;29:149-153 and U.S. Pat. Nos. 8,450,471, 8,440,431 and 8,440,432, all of which are specifically incorporated by reference. By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme Fokl. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. With respect to general information on CRISPR/Cas Systems, components thereof, and delivery of such components, including methods, materials, delivery vehicles, vectors, particles, and making and using thereof, including as to amounts and formulations, as well as Cas9CRISPR/Cas-expressing eukaryotic cells, Cas-9 CRISPR/Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); 14/054,414 European Patent Applications EP 2 771 468 (EP13818570.7), EP 2 764 103 (EP13824232.6), and EP 2 784 162 (EP14170383.5); and PCT Patent Publications wo 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and US Provisional Patent

Applications Serial Nos.: 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed April 15,2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014. Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-F EB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; US application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 June 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 June 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014,

CRISPR MEDIATED IN VIVO MODELING AND GENETIC SCREENING OF TUMOR GROWTH AND METASTASIS. Mention is made of U.S. applications 62/181,659, 18 June 2015 and 62/207,318, 19 Aug, 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 June 2015 and 62/245,264, 22-Oct-2015, NOVEL CRISPR ENZYMES AND SYSTEMS, U.S. applications 62/181,675, 18 June 2015, and Attorney Docket No. 46783.01.2128, filed 22 Oct. 2015, NOVEL CRISPR ENZYMES AND SYSTEMS, U.S. application 62/232,067, 24 Sep. 2015, U.S. application 62/205,733, 16 Aug. 2015, U.S. application 62/201,542, 5 Aug. 2015, U.S. application 62/193,507, 16 July 2015, and U.S. application 62/181,739, 18 June 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE

COMPOSITIONS WITH NEW ARCHITECTURES FOR SEQUENCE MANIPULATION. Mention is also made of PCT/US2015/045504, 15 Aug. 2015, U.S. application 62/180,699, 17 June 2015, and U.S. application 62/038,358, 17 Aug. 2014, each entitled GENOME EDITING USING CAS9 NICKASES. European patent application EP3009511.

Reference is further made to Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15;339 (6121): 819-23 (2013); RNA-guided editing of bacterial genomes using CRISPR-Cas systems. Jiang W., Bikard D., Cox D., Zhang F, Marraffini L A. Nat Biotechnol Mar;31 (3): 233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9;153 (4): 910-8 (2013); Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. 2013 Aug. 22;500 (7463): 472-6. doi: 10.1038/Nature12466. Epub 2013 Aug. 23; Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674 (13) 01015-5. (2013); DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, LA., Bao, G., & Zhang, F. Nat Biotechnol doi: 10.1038/nbt.2647 (2013); Genome engineering using the CRISPR-Cas9 system. Ran, F A., Hsu, P D., Wright, J., Agarwala, V., Scott, D A., Zhang, F. Nature Protocols Nov;8 (11): 2281-308. (2013); Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print]; Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27. (2014). 156 (5): 935-49; Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. (2014) April 20. doi: 10.1038/nbt.2889; CRISPR-Cas9 Knockin Mice for Genome Editing and Cancer Modeling, Platt et al., Cell 159 (2): 440-455 (2014) DOI: 10.1016/j.cell.2014.09.014; Development and Applications of CRISPR-Cas9 for Genome Engineering, Hsu et al, Cell 157, 1262-1278 (Jun. 5, 2014) (Hsu 2014); Genetic screens in human cells using the CRISPR/Cas9 system, Wang et al., Science. 2014 Jan. 3; 343 (6166): 80-84. doi: 10.1126/science.1246981; Rational design of highly active sgRNAs for CRISPR-Cas9-mediated gene inactivation, Doench et al., Nature Biotechnology 32 (12): 1262-7 (2014) published online 3 Sep. 2014; doi: 10.1038/nbt.3026, and In vivo interrogation of gene function in the mammalian brain using CRISPR-Cas9, Swiech et al, Nature Biotechnology 33, 102-106 (2015) published online 19 Oct. 2014; doi: 10.1038/nbt.3055, Cpf1 Is a Single RNA-Guided Endonuclease of a Class 2 CRISPR-Cas System, Zetsche et al., Cell 163, 1-13 (2015); Discovery and Functional Characterization of Diverse Class 2 CRISPR-Cas Systems, Shmakov et al., Mol Cell 60 (3): 385-397 (2015); C2c2 is a single-component programmable RNA-guided RNA-targeting CRISPR effector, Abudayyeh et al, Science (2016) published online Jun. 2, 2016 doi: 10.1126/science.aaf5573. Each of these publications, patents, patent publications, and applications, and all documents cited therein or during their prosecution (“appIn cited documents”) and all documents cited or referenced in the appIn cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cpf1).

As used herein, the term “crRNA” or “guide RNA” or “single guide RNA” or “sgRNA” or “one or more nucleic acid components” of a CRISPR/Cas locus effector protein, as applicable, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay.

A guide sequence, and hence a nucleic acid-targeting guide RNA may be selected to target any target nucleic acid sequence. The target sequence may be DNA. The target sequence may be genomic DNA. The target sequence may be mitochondrial DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (IncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and IncRNA. In some more preferred embodiments, the target sequence may be a sequence within an mRNA molecule or a pre-mRNA molecule.

In certain embodiments, the gRNA comprises a stem loop, preferably a single stem loop.

In certain embodiments, the direct repeat sequence forms a stem loop, preferably a single stem loop. In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In particular embodiments, the CRISPR/Cas system requires a tracrRNA. The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and gRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop may correspond to the tracr mate sequence, and the portion of the sequence 3′ of the loop then corresponds to the tracr sequence. In a hairpin structure the portion of the sequence 5′ of the final “N” and upstream of the loop may alternatively correspond to the tracr sequence, and the portion of the sequence 3′ of the loop corresponds to the tracr mate sequence. In alternative embodiments, the CRISPR/Cas system does not require a tracrRNA, as is known by the skilled person.

In certain embodiments, the guide RNA (capable of guiding Cas to a target locus) may comprise (1) a guide sequence capable of hybridizing to a target locus and (2) a tracr mate or direct repeat sequence (in 5′ to 3′ orientation, or alternatively in 3′ to 5′ orientation, depending on the type of Cas protein, as is known by the skilled person). In particular embodiments, the CRISPR/Cas protein is characterized in that it makes use of a guide RNA comprising a guide sequence capable of hybridizing to a target locus and a direct repeat sequence, and does not require a tracrRNA. In particular embodiments, where the CRISPR/Cas protein is characterized in that it makes use of a tracrRNA, the guide sequence, tracr mate, and tracr sequence may reside in a single RNA, i.e. an sgRNA (arranged in a 5′ to 3′ orientation or alternatively arranged in a 3′ to 5′ orientation), or the tracr RNA may be a different RNA than the RNA containing the guide and tracr mate sequence. In these embodiments, the tracr hybridizes to the tracr mate sequence and directs the CRISPR/Cas complex to the target sequence.

Typically, in the context of an endogenous nucleic acid-targeting system, formation of a nucleic acid-targeting complex (comprising a guide RNA hybridized to a target sequence and complexed with one or more nucleic acid-targeting effector proteins) results in modification (such as cleavage) of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest). The skilled person will be aware of specific cut sites for selected CRISPR/Cas systems, relative to the target sequence, which as is known in the art may be within the target sequence or alternatively 3′ or 5′ of the target sequence.

In some embodiments, the unmodified nucleic acid-targeting effector protein may have nucleic acid cleavage activity. In some embodiments, the nuclease as described herein may direct cleavage of one or both nucleic acid (DNA, RNA, or hybrids, which may be single or double stranded) strands at the location of or near a target sequence, such as within the target sequence and/or within the complement of the target sequence or at sequences associated with the target sequence. In some embodiments, the nucleic acid-targeting effector protein may direct cleavage of one or both DNA or RNA strands within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt (e.g. for Cas9, such as SaCas9 or SpCas9). In some embodiments, the cleavage may be staggered (e.g. for Cpf1), i.e. generating sticky ends. In some embodiments, the cleavage is a staggered cut with a 5′ overhang. In some embodiments, the cleavage is a staggered cut with a 5′ overhang of 1 to 5 nucleotides, preferably of 4 or 5 nucleotides. In some embodiments, the cleavage site is upstream of the PAM. In some embodiments, the cleavage site is downstream of the PAM. In some embodiments, the nucleic acid-targeting effector protein that may be mutated with respect to a corresponding wild-type enzyme such that the mutated nucleic acid-targeting effector protein lacks the ability to cleave one or both DNA or RNA strands of a target polynucleotide containing a target sequence. As a further example, two or more catalytic domains of a Cas protein (e.g. RuvC I, RuvC II, and RuvC III or the HNH domain of a Cas9 protein) may be mutated to produce a mutated Cas protein substantially lacking all DNA cleavage activity. In some embodiments, a nucleic acid-targeting effector protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “modified” Cas generally refers to a Cas protein having one or more modifications or mutations (including point mutations, truncations, insertions, deletions, chimeras, fusion proteins, etc.) compared to the wild type Cas protein from which it is derived. By derived is meant that the derived enzyme is largely based, in the sense of having a high degree of sequence homology with, a wildtype enzyme, but that it has been mutated (modified) in some way as known in the art or as described herein.

In a particular embodiment, a mutated nucleic acid-targeting effector protein based on CRISPR system as described above which lacks the ability to cleave one or both DNA or RNA strands of a target polynucleotide containing a target sequence can be fused to other tools like other nucleases, nickases, recombinases, transposases, base editors or molecular complexes including these tools. A “base editor” as used herein refers to a protein or a fragment thereof having the same catalytical activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest. Preferably, the at least one base editor in the context of the present invention is temporarily or permanently linked to at least one site-specific effector, or optionally to a component of at least one site-specific effector complex (e.g., DNA recognition domain of CRISPR system, zinc finger or TAL efectors). The linkage can be covalent and/or non-covalent.

Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalysed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA ‘R-loop’ complex

(Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. It was reasoned that a subset of this stretch of ssDNA in the R-loop might serve as an efficient substrate for a dCas9-tethered cytidine deaminase to effect direct, programmable conversion of C to U in DNA (Komor et al., supra). Recently, Goudelli et al ((2017). Programmable base editing of A·T to G·C in genomic DNA without DNA cleavage. Nature, 551 (7681), 464.) described adenine base editors (ABEs) that mediate the conversion of A·T to G·C in genomic DNA.

Any base editing complex according to the present invention can thus comprise at least one cytidine deaminase, or a catalytically active fragment thereof. The at least one base editing complex can comprise the cytidine deaminase, or a domain thereof in the form of a catalytically active fragment, as base editor.

In another embodiment, the at least one first targeted base modification is a conversion of any nucleotide C, A, T, or G, to any other nucleotide. Any one of a C, A, T or G nucleotide can be exchanged in a site-directed way as mediated by a base editor, or a catalytically active fragment thereof, to another nucleotide. The at least one base editing complex can thus comprise any base editor, or a base editor domain or catalytically active fragment thereof, which can convert a nucleotide of interest into any other nucleotide of interest in a targeted way.

In certain embodiments, the target sequence for CRISPR/Cas should be associated with a PAM (protospacer adjacent motif) or PFS (protospacer flanking sequence or site); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). Examples of PAM sequences are given in the examples section below, and the skilled person will be able to identify further PAM sequences for use with a given CRISPR enzyme. Further, engineering of the PAM Interacting (PI) domain may allow programing of PAM specificity, improve target site recognition fidelity, and increase the versatility of the Cas, e.g. Cas9, genome engineering platform. Cas proteins, such as Cas9 proteins may be engineered to alter their PAM specificity, for example as described in Kleinstiver BP et al. Engineered CRISPR-Cas9 nucleases with altered PAM specificities. Nature. 2015 Jul. 23; 523 (7561): 481-5. doi: 10.1038/nature14592. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence. The skilled person will understand that other Cas proteins may be modified analogously.

The Cas protein as referred to herein, such as without limitation Cas9, Cpf1 (Cas12a), C2c1 (Cas12b), C2c2 (Cas13a), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is (modified) Cpf1, preferably Acidaminococcus sp., such as Acidaminococcus sp. BV3L6 Cpf1 (AsCpf1) or Lachnospiraceae bacterium Cpf1, such as Lachnospiraceae bacterium MA2020 or Lachnospiraceae bacterium MD2006 (LbCpf1). In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b.

In certain embodiments, the nucleic acid modification is effected by random mutagenesis. Cells or organisms may be exposed to mutagens such as UV radiation or mutagenic chemicals (such as for instance such as ethyl methanesulfonate (EMS)), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nucleases. The products are then separated by size, such as by HPLC. See also McCallum et al. “Targeted screening for induced mutations”; Nat Biotechnol. 2000 April; 18 (4): 455-7 and McCallum et al. “Targeting induced local lesions IN genomes (TILLING) for plant functional genomics”; Plant Physiol. 2000 June; 123 (2): 439-42.

RNA interference (RNAi) is a biological process in which RNA molecules inhibit gene expression or translation, by neutralizing targeted mRNA molecules. Two types of small ribonucleic acid (RNA) molecules-microRNA (miRNA) and small interfering RNA (siRNA)-are central to RNA interference. RNAs are the direct products of genes, and these small RNAs can bind to other specific messenger RNA (mRNA) molecules and either increase or decrease their activity, for example by preventing an mRNA from being translated into a protein. The RNAi pathway is found in many eukaryotes, including animals, and is initiated by the enzyme Dicer, which cleaves long double-stranded RNA (dsRNA) molecules into short double-stranded fragments of about 21 nucleotide siRNAs (small interfering RNAs). Each siRNA is unwound into two single-stranded RNAs (ssRNAs), the passenger strand and the guide strand. The passenger strand is degraded and the guide strand is incorporated into the RNA-induced silencing complex (RISC).

Mature miRNAs are structurally similar to siRNAs produced from exogenous dsRNA, but before reaching maturity, miRNAs must first undergo extensive post-transcriptional modification. A miRNA is expressed from a much longer RNA-coding gene as a primary transcript known as a pri-miRNA which is processed, in the cell nucleus, to a 70-nucleotide stem-loop structure called a pre-miRNA by the microprocessor complex. This complex consists of an RNase III enzyme called Drosha and a dsRNA-binding protein DGCR8. The dsRNA portion of this pre-miRNA is bound and cleaved by Dicer to produce the mature miRNA molecule that can be integrated into the RISC complex; thus, miRNA and siRNA share the same downstream cellular machinery. A short hairpin RNA or small hairpin RNA (shRNA/Hairpin Vector) is an artificial RNA molecule with a tight hairpin turn that can be used to silence target gene expression via RNA interference. The most well-studied outcome is post-transcriptional gene silencing, which occurs when the guide strand pairs with a complementary sequence in a messenger RNA molecule and induces cleavage by Argonaute 2 (Ago2), the catalytic component of the RISC. As used herein, an RNAi molecule may be an siRNA, shRNA, or a miRNA. In will be understood that the RNAi molecules can be applied as such to/in the plant, or can be encoded by appropriate vectors, from which the RNAi molecule is expressed. Delivery and expression systems of RNAi molecules, such as siRNAs, shRNAs or miRNAs are well known in the art.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. As used herein, the term “heterozygote” refers to an individual cell or plant having different alleles at one or more or all loci. When the term is used with reference to a specific locus or gene, it means at least that locus or gene has different alleles. As used herein, the term “heterozygous” means a genetic condition existing when different alleles reside at corresponding loci on homologous chromosomes. In certain embodiments, the QTL and/or one or more marker(s) as described herein is/are homozygous. In certain embodiments, the QTL and/or one or more marker(s) as described herein are heterozygous. In certain embodiments, the QTL allele and/or one or more marker(s) allele(s) as described herein is/are homozygous. In certain embodiments, the QTL allele and/or one or more marker(s) allele(s) as described herein are heterozygous.

A “marker” is a (means of finding a position on a) genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. The term marker locus is the locus (gene, sequence or nucleotide) that the marker detects. “Marker” or “molecular marker” or “marker locus” may also be used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population. With regard to a SNP marker, allele refers to the specific nucleotide base present at that SNP locus in that individual plant.

“Fine-mapping” refers to methods by which the position of a QTL can be determined more accurately (narrowed down) and by which the size of the introgression fragment comprising the QTL is reduced. For example Near Isogenic Lines for the QTL (QTL-NILS) can be made, which contain different, overlapping fragments of the introgression fragment within an otherwise uniform genetic background of the recurrent parent. Such lines can then be used to map on which fragment the QTL is located and to identify a line having a shorter introgression fragment comprising the QTL.

“Marker assisted selection” (of MAS) is a process by which individual plants are selected based on marker genotypes. “Marker assisted counter-selection” is a process by which marker genotypes are used to identify plants that will not be selected, allowing them to be removed from a breeding program or planting. Marker assisted selection uses the presence of molecular markers, which are genetically linked to a particular locus or to a particular chromosome region (e.g. introgression fragment, transgene, polymorphism, mutation, etc), to select plants for the presence of the specific locus or region (introgression fragment, transgene, polymorphism, mutation, etc). For example, a molecular marker genetically linked to a digestibility QTL as defined herein, can be used to detect and/or select plants comprising the QTL on chromosome 9. The closer the genetic linkage of the molecular marker to the locus (e.g. about 7 cM, 6 CM, 5 CM, 4 CM, 3 cM, 2 cM, 1 cM, 0.5 CM or less), the less likely it is that the marker is dissociated from the locus through meiotic recombination. Likewise, the closer two markers are linked to each other (e.g. within 7 cM or 5 CM, 4 CM, 3 cM, 2 cM, 1 cM or less) the less likely it is that the two markers will be separated from one another (and the more likely they will co-segregate as a unit). “LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favor the presence of linkage and a LOD score greater than 3.0 is considered evidence for linkage. A LOD score of +3 indicates 1000 to 1 odds that the linkage being observed did not occur by chance. A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

“Genetic markers” are nucleic acids that are polymorphic in a population and where the alleles of which can be detected and distinguished by one or more analytic methods, e.g., RFLP, AFLP, isozyme, SNP, SSR, and the like. The terms “molecular marker” and “genetic marker” are used interchangeably herein. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms

(AFLPs). Well established methods are also know for the detection of expressed sequence tags (ESTs) and SSR markers derived from EST sequences and randomly amplified polymorphic DNA (RAPD).

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”. The term “indel” refers to an insertion or deletion, wherein one line may be referred to as having an inserted nucleotide or piece of DNA relative to a second line, or the second line may be referred to as having a deleted nucleotide or piece of DNA relative to the first line.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (CM). One cM corresponds to a recombination frequency of 1%. If no recombinants can be found, the RF is zero and the loci are either extremely close together physically or they are identical. The further apart two loci are, the higher the RF.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

An allele “negatively” correlates with a trait when it is linked to it and when presence of the allele is an indicator that a desired trait or trait form will not occur in a plant comprising the allele. An allele “positively” correlates with a trait when it is linked to it and when presence of the allele is an indicator that the desired trait or trait form will occur in a plant comprising the allele.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to gray leaf spot). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

“Linkage” refers to the tendency for alleles to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a genetic map distance of 1.0 centiMorgan (1.0 cM). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

As used herein, the term “sequence identity” refers to the degree of identity between any given nucleic acid sequence and a target nucleic acid sequence. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences.

Percent sequence identity also can be determined for any amino acid sequence. To determine percent sequence identity, a target nucleic acid or amino acid sequence is compared to the identified nucleic acid or amino acid sequence using the BLAST 2 Sequences (BI2seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the BI2seq program can be found in the readme file accompanying BLASTZ. B12seq performs a comparison between two sequences using either the BLASTN or BLASTP algorithm.

BLASTN is used to compare nucleic acid sequences, while BLASTP is used to compare amino acid sequences. To compare two nucleic acid sequences, the options are set as follows:-i is set to a file containing the first nucleic acid sequence to be compared (e.g., C:544 seq I.txt);-j is set to a file containing the second nucleic acid sequence to be compared (e.g., C:\seq2.txt); -p is set to blastn;-o is set to any desired file name (e.g., C:\output.txt);-q is set to-1;-r is set to 2; and all other options are left at their default setting. The following command will generate an output file containing a comparison between two sequences: C:\B12seq-i c:\seql.txt-j c:\seq2.txt-p blastn-o c:\output.txt-q-1-r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the BI2seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

When reference is made to a nucleic acid sequence (e.g. DNA or genomic DNA) having “substantial sequence identity to” a reference sequence or having a sequence identity of at least 60%, e.g. at least 70%, 75%, 80%, 85%, 90%, 95%, 98% or 99% nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically, stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2X SSC at 63° C. for 20 min, or equivalent conditions. Stringent conditions for DNA-DNA hybridisation (Southern blots using a probe of e.g. 100 nt) are for example those which include at least one wash (usually 2) in 0.2X SSC at a temperature of at least 50° C., usually about 55° C., for 20 min, or equivalent conditions. See also Sambrook et al. (1989) and Sambrook and Russell (2001). Examples of high stringent hybridization conditions are conditions under which primarily only those nucleic acid molecules that have at least 90% or at least 95% sequence identity undergo hybridization. Such high stringent hybridization conditions are, for example: 4×SSC at 65° C. and subsequent multiple washes in 0.1×SSC at 65° C. for approximately 1 hour. The term “high stringent hybridization conditions” as used herein may also mean: hybridization at 68° C. in 0.25 M sodium phosphate, pH 7.2, 7% SDS, 1 mM EDTA and 1% BSA for 16 hours and subsequently washing twice with 2×SSC and 0.1% SDS at 68° C. Preferably, hybridization takes place under stringent conditions. Less stringent hybridization conditions are, for example: hybridizing in 4×SSC at 37° C. and subsequent multiple washing in 1 x SSC at room temperature.

As used herein, F35H (ExPASy enzyme entry EC 1.14.13.88) refers to the flavonoid 3′,5′-hydroxylase gene or protein. F35H is also known as F3′5′H, F3′,5′H, cytochrome P450 flavonoid 3′,5′-hydroxylase, or flavanone, NADPH: oxygen oxidoreductase. F35H catalyzes the following reaction: flavanone+2 NADPH+2 O(2)<=>3′,5′-dihydroxyflavanone+2 NADP (+)+2 H (2) O.

In an aspect, the invention relates to a method for identifying a plant or plant part having improved digestibility, preferably improved stover digestibility, or for selecting a plant or plant part having improved digestibility, preferably improved stover digestibility, comprising screening for the presence of a QTL allele (such as in isolated genetic material from the plant or plant part) associated with improved digestibility, preferably improved stover digestibility, said QTL allele comprising a nucleotide sequence of a gene encoding a cytochrome P450 flavonoid 3′,5′-hydroxylase (F35H), having a mutation.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, and screening in said genetic material for the presence of a QTL allele associated with improved digestibility, preferably improved stover digestibility, said QTL allele comprising a nucleotide sequence of a gene encoding a cytochrome P450 flavonoid 3′,5′-hydroxylase (F35H), having a mutation.

In an embodiment, the invention relates to a method for identifying a plant or plant part having improved digestibility, preferably improved stover digestibility, or for selecting a plant or plant part having improved digestibility, preferably improved stover digestibility, comprising screening for the presence of a QTL allele (such as in isolated genetic material from the plant or plant part) associated with improved digestibility, preferably improved stover digestibility, said QTL allele comprising a nucleotide sequence of a gene encoding a cytochrome P450 flavonoid 3′,5′-hydroxylase (F35H) having a mutation, and selecting a plant or plant part in which the QTL allele is present.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, screening in said genetic material for the presence of a QTL allele associated with improved digestibility, preferably improved stover digestibility, said QTL allele comprising a nucleotide sequence of a gene encoding a cytochrome P450 flavonoid 3′,5′-hydroxylase (F35H), having a mutation, and selecting a plant or plant part in which the QTL allele is present.

In an aspect, the invention relates to a method for identifying a plant or plant part having improved digestibility, preferably improved stover digestibility, or for selecting a plant or plant part having improved digestibility, preferably improved stover digestibility, comprising screening for the presence of a QTL allele (such as in isolated genetic material from the plant or plant part) comprising a nucleotide sequence of a gene encoding a cytochrome P450 flavonoid 3′,5′-hydroxylase (F35H), having a mutation.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, and screening in said genetic material for the presence of a QTL allele comprising a nucleotide sequence of a gene encoding a cytochrome P450 flavonoid 3′,5′-hydroxylase (F35H), having a mutation.

In an embodiment, the invention relates to a method for identifying a plant or plant part having improved digestibility, preferably improved stover digestibility, or for selecting a plant or plant part having improved digestibility, preferably improved stover digestibility, comprising screening for the presence of a QTL allele (such as in isolated genetic material from the plant or plant part) comprising a nucleotide sequence of a gene encoding a cytochrome P450 flavonoid 3′,5′-hydroxylase (F35H) having a mutation, and selecting a plant or plant part in which the QTL allele is present.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, screening in said genetic material for the presence of a QTL allele comprising a nucleotide sequence of a gene encoding a cytochrome P450 flavonoid 3′,5′-hydroxylase (F35H) having a mutation, and selecting a plant or plant part in which the QTL allele is present.

In certain embodiments, the plant is maize and the QTL allele is located on chromosome 9 and comprises and/or is flanked by (molecular) marker alleles ma61070s01 and ma30168s02, preferably by marker alleles ma50827s01 and ma16983s02, more preferably by marker alleles ma17117s01 and ma61125s01.

In certain embodiments, the plant is maize and the QTL allele is located on chromosome 9 and comprises the (molecular) marker allele of ma61134xxx.

In certain embodiments, the plant is maize and the QTL allele is located on a chromosomal interval comprising and/or flanked by (molecular) marker alleles ma61070s01 and ma30168s02,, preferably by marker alleles ma50827s01 and ma16983s02, more preferably by marker alleles ma17117s01 and ma61125s01.

In certain embodiments, the plant is maize and the QTL allele is located on a chromosomal interval comprising the marker allele of ma61134xxx and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

In an aspect, the invention relates to a method for identifying a plant or plant part having improved digestibility, preferably improved stover digestibility, or for selecting a plant or plant part having improved digestibility, preferably improved stover digestibility, comprising screening (such as in isolated genetic material from the plant or plant part) for the presence of a mutation in a nucleotide sequence of a gene encoding a cytochrome P450 flavonoid 3′,5′-hydroxylase (F35H) or for the presence of a mutation leading to altered expression of the mRNA of a gene encoding a cytochrome P450 flavonoid 3′,5′-hydroxylase (F35H) and/or the F35H protein, or a mutation, preferably a mutation leading to an F35H protein having altered enzymatic activity upon translation, preferably leading to reduced or absent expression of the mRNA of a gene encoding a cytochrome P450 flavonoid 3′,5′-hydroxylase (F35H) and/or the F35H protein (such as a knock-down or knock-out mutation), or a mutation, preferably a mutation leading to a non-functional F35H protein or an F35H protein having reduced enzymatic activity upon translation or an F35H protein having increased enzymatic activity upon translation.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, and screening in said genetic material for the presence of a mutation in a nucleotide sequence of a gene encoding a cytochrome P450 flavonoid 3′,5′-hydroxylase (F35H) or for the presence of a mutation leading to altered expression of the mRNA of a gene encoding a cytochrome P450 flavonoid 3′,5′-hydroxylase (F35H) and/or the F35H protein, or a mutation, preferably a mutation leading to an F35H protein having altered enzymatic activity upon translation, preferably leading to reduced or absent expression of the mRNA of a gene encoding a cytochrome P450 flavonoid 3′,5′-hydroxylase (F35H) and/or the F35H protein (such as a knock-down or knock-out mutation), or a mutation, preferably a mutation leading to a non-functional F35H protein or an F35H protein having reduced enzymatic activity upon translation or an F35H protein having increased enzymatic activity upon translation.

In an embodiment, the invention relates to a method for identifying a plant or plant part having improved digestibility, preferably improved stover digestibility, or for selecting a plant or plant part having improved digestibility, preferably improved stover digestibility, comprising screening (such as in isolated genetic material from the plant or plant part) for the presence of a mutation in a nucleotide sequence of a gene encoding a cytochrome P450 flavonoid 3′,5′-hydroxylase (F35H), or for the presence of a mutation leading to altered expression of the mRNA of a gene encoding a cytochrome P450 flavonoid 3′,5′-hydroxylase (F35H) and/or the F35H protein, or a mutation, preferably a mutation leading to an F35H protein having altered enzymatic activity upon translation, preferably leading to reduced or absent expression of the mRNA of a gene encoding a cytochrome P450 flavonoid 3′,5′-hydroxylase (F35H) and/or the F35H protein (such as a knock-down or knock-out mutation), or a mutation, preferably a mutation leading to a non-functional F35H protein or an F35H protein having reduced enzymatic activity upon translation or an F35H protein having increased enzymatic activity upon translation, and selecting a plant or plant part in which the mutation in said F35H is present.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, screening in said genetic material for the presence of a mutation in a nucleotide sequence of a gene encoding a cytochrome P450 flavonoid 3′,5′-hydroxylase (F35H), or for the presence of a mutation leading to altered expression of the mRNA of a gene encoding a cytochrome P450 flavonoid 3′,5′-hydroxylase (F35H) and/or the F35H protein, or a mutation, preferably a mutation leading to an F35H protein having altered enzymatic activity upon translation, preferably leading to reduced or absent expression of the mRNA of a gene encoding a cytochrome P450 flavonoid 3′,5′-hydroxylase (F35H) and/or the F35H protein (such as a knock-down or knock-out mutation), or a mutation, preferably a mutation leading to a non-functional F35H protein or an F35H protein having reduced enzymatic activity upon translation or an F35H protein having increased enzymatic activity upon translation, and selecting a plant or plant part in which a mutation in said F35H is present.

In an aspect, the invention relates to a method for identifying a plant or plant part having improved digestibility, preferably improved stover digestibility, or for selecting a plant or plant part having improved digestibility, preferably improved stover digestibility, comprising (such as in isolated material from the plant or plant part) analysing the (protein and/or mRNA) expression level of a gene encoding a cytochrome P450 flavonoid 3′,5′-hydroxylase (F35H) and/or screening for altered expression of the mRNA of a gene encoding a cytochrome P450 flavonoid 3′,5′-hydroxylase (F35H) and/or the F35H protein or an F35H protein having altered enzymatic activity, preferably for reduced or absent expression of the mRNA of a gene encoding a cytochrome P450 flavonoid 3′,5′-hydroxylase (F35H) and/or the F35H protein or for a non-functional F35H protein or an F35H protein having reduced enzymatic activity or an F35H protein having reduced enzymatic activity.

In an embodiment, the invention relates to a method for identifying a plant or plant part having improved digestibility, preferably improved stover digestibility, or for selecting a plant or plant part having improved digestibility, preferably improved stover digestibility, comprising isolating material from at least one cell of the plant or plant part, and analysing the (protein and/or mRNA) expression level of a gene encoding a cytochrome P450 flavonoid 3′,5′-hydroxylase (F35H) in said material and/or screening for altered expression of the mRNA of a gene encoding a cytochrome P450 flavonoid 3′,5′-hydroxylase (F35H) and/or the F35H protein or an F35H protein having altered enzymatic activity, preferably for reduced or absent expression of the mRNA of a gene encoding a cytochrome P450 flavonoid 3′,5′-hydroxylase (F35H) and/or the F35H protein or for a non-functional F35H protein or an F35H protein having reduced enzymatic activity or an F35H protein having reduced enzymatic activity in said material.

In an embodiment, the invention relates to a method for identifying a plant or plant part having improved digestibility, preferably improved stover digestibility, or for selecting a plant or plant part having improved digestibility, preferably improved stover digestibility, comprising analysing (such as in isolated material from the plant or plant part) the (protein and/or mRNA) expression level of a gene encoding a cytochrome P450 flavonoid 3′,5′-hydroxylase (F35H), and selecting a plant or plant part in which the F35H mRNA and/or protein expression or the enzymatic F35H activity is altered, preferably is reduced or eliminated or the enzymatic F35H activity is reduced or increased.

In an embodiment, the invention relates to a method for identifying a plant or plant part having improved digestibility, preferably improved stover digestibility, or for selecting a plant or plant part having improved digestibility, preferably improved stover digestibility, comprising isolating material from at least one cell of the plant or plant part, analysing the (protein and/or mRNA) expression level of a gene encoding a cytochrome P450 flavonoid 3′,5′-hydroxylase (F35H) in said material and/or screening for altered expression of the mRNA of a gene encoding a cytochrome P450 flavonoid 3′,5′-hydroxylase (F35H) and/or the F35H protein or an F35H protein having altered enzymatic activity, preferably for reduced or absent expression of the mRNA of a gene encoding a cytochrome P450 flavonoid 3′,5′-hydroxylase (F35H) and/or the F35H protein or for a non-functional F35H protein or an F35H protein having reduced enzymatic activity or an F35H protein having reduced enzymatic activity in said material, and selecting a plant or plant part in which the F35H mRNA and/or protein expression or the enzymatic F35H activity is altered, preferably reduced or eliminated or the enzymatic

F35H activity is reduced or increased.

In certain embodiments, if the (protein and/or mRNA) expression level of the F35H, in particular the wild type or native F35H, is altered, then the plant or plant part has improved digestibility. In certain embodiments, if the (protein and/or mRNA) expression level of the F35H, in particular the wild type or native F35H, is altered compared to a reference expression level, then the plant or plant part has improved digestibility. In certain embodiments, if the (protein and/or mRNA) expression level of the F35H, in particular the wild type or native F35H, is altered compared to the reference expression level in a reference plant or plant part, then the plant or plant part has improved digestibility. In certain embodiments, the reference plant (or plant part) is the maize inbred line PH207, as described in “Draft Assembly of Elite Inbred Line PH207 Provides Insights into Genomic and Transcriptome Diversity in Maize”, Hirsch et al., Plant Cell. 2016 November; 28 (11): 2700-2714. Published online 2016 Nov. 1. doi: 10.1105/tpc.16.00353, or a maize plant comprising the QTL allele comprising the wild type or native (unmutated) nucleotide sequence of a gene encoding a cytochrome P450 flavonoid 3′,5′-hydroxylase (F35H) (e.g., derived from PH207), preferably the reference plant (or plant part) is derived from a near isogenic line.

As used herein, altered (protein and/or mRNA) expression levels refers to increased or decreased expression levels of about at least 10%, preferably at least 30%, more preferably at least 50%, such as at least 20%, 40%, 60%, 80% or more, such as at least 85%, at least 90%, at least 95%, or more.

In a particular embodiments, if the (protein and/or mRNA) expression level of the F35H, in particular the wild type or native F35H, is reduced or expression is (substantially) absent or eliminated, then the plant or plant part has improved digestibility. In certain embodiments, if the (protein and/or mRNA) expression level of the F35H, in particular the wild type or native F35H, is reduced or expression is (substantially) absent or eliminated compared to a reference expression level, then the plant or plant part has improved digestibility. In certain embodiments, if the (protein and/or mRNA) expression level of the F35H, in particular the wild type or native F35H, is reduced or expression is (substantially) absent or eliminated compared to the reference expression level in a reference plant or plant part, then the plant or plant part has improved digestibility. In certain embodiments, the reference plant (or plant part) is the maize inbred line PH207, as described in “Draft Assembly of Elite Inbred Line PH207 Provides Insights into Genomic and Transcriptome Diversity in Maize”, Hirsch et al., Plant Cell. 2016 November; 28 (11): 2700-2714. Published online 2016 Nov. 1. doi: 10.1105/tpc.16.00353, or a maize plant comprising the QTL allele comprising the wild type or native (unmutated) nucleotide sequence of a gene encoding a cytochrome P450 flavonoid 3′,5′-hydroxylase (F35H) (e.g., derived from PH207), preferably the reference plant (or plant part) is derived from a near isogenic line.

As used herein, reduced (protein and/or mRNA) expression levels refers to decreased expression levels of about at least 10%, preferably at least 30%, more preferably at least 50%, such as at least 20%, 40%, 60%, 80% or more, such as at least 85%, at least 90%, at least 95%, or more. Expression is (substantially) absent or eliminated if expression levels are reduced at least 80%, preferably at least 90%, more preferably at least 95%. In certain embodiments, expression is (substantially) absent, if no protein and/or mRNA, in particular the wild type or native protein and/or mRNA, can be detected, such as by standard detection methods, including for instance (quantitative) PCR, northern blot, western blot, ELISA, etc.

In an aspect, the invention relates to a method for identifying a plant or plant part having improved digestibility, preferably improved stover digestibility, or for selecting a plant or plant part having improved digestibility, preferably improved stover digestibility, comprising screening (such as in isolated genetic material from the plant or plant part) for the presence of one or more (molecular) marker allele associated with improved digestibility, said (molecular) marker allele being the molecular marker allele of ma61134xxx, and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, and screening in said genetic material for the presence of one or more (molecular) marker allele associated with improved digestibility, said (molecular) marker allele being the molecular marker allele of ma61134xxx, and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

In an embodiment, the invention relates to a method for identifying a plant or plant part having improved digestibility, preferably improved stover digestibility, or for selecting a plant or plant part having improved digestibility, preferably improved stover digestibility, comprising screening (such as in isolated genetic material from the plant or plant part) for the presence of one or more (molecular) marker allele associated with improved digestibility, said (molecular) marker allele being the molecular marker allele of ma61134xxx, and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility, and selecting a plant or plant part in which the one or more (molecular) marker allele is present.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, screening in said genetic material for the presence of one or more (molecular) marker allele associated with improved digestibility, said (molecular) marker allele being the molecular marker allele of ma61134xxx, and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility, and selecting a plant or plant part in which the one or more (molecular) marker allele is present.

In an aspect, the invention relates to a method for identifying a plant or plant part having improved digestibility, preferably improved stover digestibility, or for selecting a plant or plant part having improved digestibility, preferably improved stover digestibility, comprising screening (such as in isolated genetic material from the plant or plant part) for the presence of the molecular marker allele of ma61134xxx, and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, and screening in said genetic material for the presence of the molecular marker allele of ma61134xxx, and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility. In an embodiment, the invention relates to a method for identifying a plant or plant part having improved digestibility, preferably improved stover digestibility, or for selecting a plant or plant part having improved digestibility, preferably improved stover digestibility, comprising screening (such as in isolated genetic material from the plant or plant part) for the presence of the molecular marker allele of ma61134xxx, and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility, and selecting a plant or plant part in which the one or more (molecular) marker allele is present.

In an embodiment, the invention relates to a method for identifying a plant or plant part having improved digestibility, preferably improved stover digestibility, or for selecting a plant or plant part having improved digestibility, preferably improved stover digestibility, comprising isolating genetic material from at least one cell of the plant or plant part, screening in said genetic material for the presence of the molecular marker allele of ma61134xxx, and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility, and selecting a plant or plant part in which the one or more (molecular) marker allele is present.

It will be understood that in the methods as described above, when the QTL allele or (molecular) marker allele is present, then the plant or plant part is identified as having improved digestibility.

Methods for screening for the presence of a QTL allele or (molecular) marker allele as described herein are known in the art. Without limitation, screening may encompass or comprise sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay), etc.

In an aspect, the invention relates to a method, such as a method for generating/producing a plant or plant part having improved digestibility, preferably improved stover digestibility, and/or improving digestibility of a plant or plant part, preferably stover digestibility, comprising introducing or introgressing into the genome of a plant or plant part a QTL allele associated with improved digestibility and comprising a nucleotide sequence of a gene encoding a cytochrome P450 flavonoid 3′,5′-hydroxylase having a mutation.

In an aspect, the invention relates to a method, such as a method for generating/producing a plant or plant part having improved digestibility, preferably improved stover digestibility, and/or for improving digestibility of a plant or plant part, preferably stover digestibility, comprising introducing or introgressing into the genome of a plant or plant part a QTL allele comprising a nucleotide sequence of a gene encoding a cytochrome P450 flavonoid 3′,5′-hydroxylase having a mutation.

In certain embodiments, the mutation leads to altered expression of the mRNA of the gene and/or the F35H protein, or the mutation leads to an F35H protein having altered enzymatic activity upon translation, more preferably the mutation leads to reduced or absent expression of the mRNA of said gene and/or the F35H protein, to a knock-out or knock-down of said gene or a mutation leading to a non-functional F35H protein (e.g., truncated F35H protein) (e.g., truncated F35H protein) or an F35H protein having reduced enzymatic activity upon translation or an F35H protein having increased enzymatic activity upon translation.

In certain embodiments of both aforementioned aspects related to the method for generating/producing a plant or plant part and/or for improving digestibility of a plant or plant part, preferably stover digestibility, the QTL allele is located on chromosome 9 and comprises and/or is flanked by marker alleles ma61070s01 and ma30168s02, preferably by marker alleles ma50827s01 and ma16983s02, more preferably by marker alleles ma17117s01 and ma61125s01.

In certain embodiments, the QTL allele is located on chromosome 9 and comprises the (molecular) marker allele of ma61134xxx.

In certain embodiments, the QTL allele is located on a chromosomal interval comprising and/or flanked by (molecular) marker alleles ma61070s01 and ma30168s02, preferably by marker alleles ma50827s01 and ma16983s02, more preferably by marker alleles ma17117s01 and ma61125s01.

In certain embodiments, the QTL allele is located on a chromosomal interval comprising the marker allele of ma61134xxx and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

In an aspect, the invention relates to a method, such as a method for generating/producing a plant or plant part having improved digestibility, preferably improved stover digestibility, and/or for improving digestibility of a plant or plant part, preferably stover digestibility, comprising introducing into the genome of a plant or plant part a QTL allele associated with improved digestibility and comprising the marker allele of ma61134xxx and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

In an aspect, the invention relates to a method, such as a method for generating/producing a plant or plant part having improved digestibility, preferably improved stover digestibility, and/or for improving digestibility of a plant or plant part, preferably stover digestibility, comprising introducing into the genome of a plant or plant part a QTL allele comprising the marker allele of ma61134xxx and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

In an aspect, the invention relates to a method, such as a method for generating/producing a plant or plant part having improved digestibility, preferably improved stover digestibility, and/or for improving digestibility of a plant or plant part, preferably stover digestibility, comprising introducing or introgressing into the genome of a plant or plant part a nucleotide sequence of a gene encoding a cytochrome P450 flavonoid 3′,5′-hydroxylase (F35H) having a mutation.

In an aspect, the invention relates to a method, such as a method for generating/producing a plant or plant part having improved digestibility, preferably improved stover digestibility, and/or for improving digestibility of a plant or plant part, preferably stover digestibility, comprising introducing into the genome of a plant or plant, in particular into a nucleotide sequence of an endogenous gene encoding a cytochrome P450 flavonoid 3′,5′-hydroxylase a mutation.

In an aspect, the invention relates to a method, such as a method for generating/producing a plant or plant part having improved digestibility, preferably improved stover digestibility, and/or for improving digestibility of a plant or plant part, preferably stover digestibility, comprising altering an endogenous gene encoding a cytochrome P450 flavonoid 3′,5′-hydroxylase gene, preferably knocking out an endogenous gene encoding a cytochrome P450 flavonoid 3′,5′-hydroxylase gene.

In an aspect, the invention relates to a method, such as a method for generating/producing a plant or plant part having improved digestibility, preferably improved stover digestibility, and/or for improving digestibility of a plant or plant part, preferably stover digestibility, comprising altering mRNA expression of gene encoding a cytochrome P450 flavonoid 3′,5′-hydroxylase gene (F35H) and/or the encoded F35H protein, preferably knocking down mRNA expression of gene encoding a cytochrome P450 flavonoid 3′,5′-hydroxylase gene (F35H) and/or the encoded F35H protein. In an aspect, the invention relates to a method, such as a method for generating/producing a plant or plant part having improved digestibility, preferably improved stover digestibility, and/or for improving digestibility of a plant or plant part, preferably stover digestibility, comprising altering the expression of mRNA of a gene encoding a cytochrome P450 flavonoid 3′,5′-hydroxylase and/or the F35H protein or altering the enzymatic activity of a cytochrome P450 flavonoid 3′,5′-hydroxylase (F35H), preferably eliminating or reducing or inhibiting expression of mRNA of a gene encoding a cytochrome P450 flavonoid 3′,5′-hydroxylase and/or the F35H protein, reducing the enzymatic activity of a cytochrome P450 flavonoid 3′,5′-hydroxylase (F35H) or inhibiting the F35H protein, or increasing the enzymatic activity of a cytochrome P450 flavonoid 3′,5′-hydroxylase (F35H).

In certain embodiments, the invention relates to a method, such as a method generating/producing a plant or plant part having improved digestibility, preferably improved stover digestibility, and/or for improving digestibility, preferably stover digestibility, of a plant or plant part, comprising
A. introducing into a nucleotide sequence of an endogenous gene of the plant or plant part encoding a cytochrome P450 flavonoid 3′,5′-hydroxylase a mutation or other genetic event leading to altered expression of the mRNA of the gene and/or the F35H protein, or a mutation leading to an F35H protein having altered enzymatic activity upon translation, more preferably a mutation leading reduced or absent expression of the mRNA of said gene and/or the F35H protein, to a knock-out or knock-down of said gene or a mutation leading to a non-functional F35H protein (e.g., truncated F35H protein) or an F35H protein having reduced enzymatic activity upon translation, or an F35H protein having increased enzymatic activity upon translation; or
B. introducing into the genome of the plant or plant part a first double-stranded DNA and a second double-stranded DNA, wherein the nucleotide sequences of the coding strands of the first and second DNA are reverse complements of each other, so that a transcript of the first DNA and a transcript of the second DNA are capable of hybridizing to form a double-stranded RNA, wherein the coding strand of the first or the second DNA comprises:
   a. at least 19 successive nucleotides of the nucleotide sequence of SEQ ID NO: 2, 5, or 8 or of a nucleotide sequence having at least 60% identity, preferably at least 70% or at least 80%, more preferably at least 90%, at least 92%, at least 94%, at least 95%, at least 96%, at least 97%, at least 98% or at least 99% to the sequence of SEQ ID NO: 2, 5, or 8; or
   b. a nucleotide sequence which is complementary to at least 19 successive nucleotides of the nucleotide sequence of SEQ ID NO: 2, 5, or 8 or of a nucleotide sequence having at least 60% identity, preferably at least 70% or at least 80%, more preferably at least 90%, at least 92%, at least 94%, at least 95%, at least 96%, at least 97%, at least 98% or at least 99% to the sequence of SEQ ID NO: 2, 5, or 8; or
C. a double-stranded RNA, wherein one strand corresponds to:
   a. at least 19 successive nucleotides of the nucleotide sequence of SEQ ID NO: 2,5, or 8 or of a nucleotide sequence having at least 60% identity, preferably at least 70% or at least 80%, more preferably at least 90%, at least 92%, at least 94%, at least 95%, at least 96%, at least 97%, at least 98% or at least 99% to the sequence of SEQ ID NO: 2, 5, or 8, wherein T is replaced by U; or
   b. a nucleotide sequence which is complementary to at least 19 successive nucleotides of the nucleotide sequence of SEQ ID NO: 2, 5, or 8 or of a nucleotide sequence having at least 60% identity, preferably at least 70% or at least 80%, more preferably at least 90%, at least 92%, at least 94%, at least 95%, at least 96%, at least 97%, at least 98% or at least 99% to the sequence of SEQ ID NO: 2, 5, or 8, wherein T is replaced by U; or
   D. introducing into the plant or the plant part an RNA-specific CRISPR/Cas system, such as a CRISPR/Cas13a system, directed against or targeting a nucleotide sequence encoding the F35H protein, or into the genome of the plant or plant part one or more polynucleotide sequence(s) encoding (and expressing or being capable of expressing) said RNA-specific CRISPR/Cas system; or
   E. introducing into the plant or the plant part a chemical compound or an antibody altering (or being capable to alter) the enzymatic activity of the F35H protein upon interaction with said F35H, preferably reducing (or being capable to reduce) the enzymatic activity of the F35H protein or inhibiting (or being capable to inhibit) the enzymatic activity of F35H protein or increasing (or being capable to increase) the enzymatic activity of the F35H protein upon interaction with said F35H.

In certain embodiments, the invention relates to a method, such as a method generating/producing a plant or plant part having improved digestibility, preferably improved stover digestibility, and/or for improving digestibility, preferably stover digestibility, of a plant or plant part, comprising regenerating a plant from the plant part of aforementioned modified plants or plant parts.

In an aspect, the invention relates to a plant or plant part comprising a QTL allele associated with improved digestibility, preferably improved stover digestibility, said QTL allele comprising a nucleotide sequence of a gene encoding a cytochrome P450 flavonoid 3′,5′-hydroxylase (F35H) having a mutation.

In an aspect, the invention relates to a plant or plant part comprising a QTL allele comprising a nucleotide sequence of a gene encoding a cytochrome P450 flavonoid 3′,5′-hydroxylase (F35H) having a mutation.

In certain embodiments, the mutation leads to altered expression of the mRNA of said gene and/or the F35H protein, or the mutation leads to an F35H protein having altered enzymatic activity upon translation. Altered expression of F35H may be effected for instance by any of the mutagenesis methods described herein.

In certain embodiments, the mutation leads to reduced or absent expression of the mRNA of said gene and/or the F35H protein, to a knock-out or knock-down of said gene or a mutation leading to a non-functional F35H protein (e.g., truncated F35H protein) or an F35H protein having reduced or increased enzymatic activity upon translation.

Knockdown or knockout of F35H may be effected for instance by any of the mutagenesis methods described herein.

In certain embodiments, the QTL allele is located on chromosome 9 and comprises and/or is flanked by (molecular) marker alleles ma61070s01 and ma30168s02, preferably by marker alleles ma50827s01 and ma16983s02, more preferably by marker alleles ma17117s01 and ma61125s01.

In certain embodiments, the QTL allele is located on chromosome 9 and comprises the marker allele of ma61134xxx and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

In certain embodiments, the QTL allele is located on a chromosomal interval comprising and/or flanked by (molecular) marker alleles ma61070s01 and ma30168s02, preferably by marker alleles ma50827s01 and ma16983s02, more preferably by marker alleles ma17117s01 and ma61125s01.

In certain embodiments, the QTL allele is located on a chromosomal interval comprising the marker allele of ma61134xxx and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility.

In an aspect, the invention relates to a plant or plant part comprising a nucleotide sequence of a gene encoding a cytochrome P450 flavonoid 3′,5′-hydroxylase (F35H) having a mutation.

In an aspect, the invention relates to a plant or plant part comprising the marker allele of ma61134xxx and/or one or more molecular marker alleles located in a chromosomal interval on chromosome 9 flanked by marker alleles ma61070s01 and ma30168s02, preferably by marker alleles ma50827s01 and ma16983s02, more preferably by marker alleles ma17117s01 and ma61125s01, preferably wherein the one or more molecular marker alleles are detectable by a polynucleic acid, such as an allele specific polynucleic acid (molecular marker), suitable for hybridization as forward primer and reverse primer to a locus in the chromosomal interval which co-segregates with the improved digestibility. In an aspect, the invention relates to a plant or plant part comprising
A. a gene encoding a cytochrome P450 flavonoid 3′,5′-hydroxylase (F35H) having a mutation or other genetic event, preferably a mutation or other genetic event leading to altered expression of the mRNA of the gene and/or the F35H protein, or a mutation leading to an F35H protein having altered enzymatic activity upon translation, more preferably a mutation leading to a knock-out or knock-down of said gene, or having reduced or eliminated mRNA and/or protein expression of an F35H gene, or a mutation or other genetic event leading to a non-functional F35H protein (e.g., truncated F35H protein) or an F35H protein having reduced enzymatic activity upon translation or an F35H protein having increased enzymatic activity upon translation; or
B. a (stably integrated) first double-stranded DNA and a (stably integrated) second double-stranded DNA, wherein the nucleotide sequences of the coding strands of the first and second DNA are reverse complements of each other, so that a transcript of the first DNA and a transcript of the second DNA are capable of hybridizing to form a double-stranded RNA, wherein the coding strand of the first or the second DNA comprises:
   a. at least 19 successive nucleotides of the nucleotide sequence of SEQ ID NO: 2, 5, or 8 or of a nucleotide sequence having at least 60% identity, preferably at least 70% or at least 80%, more preferably at least 90%, at least 92%, at least 94%, at least 95%, at least 96%, at least 97%, at least 98% or at least 99% to the sequence of SEQ ID NO: 2, 5, or 8, or
   b. a nucleotide sequence which is complementary to at least 19 successive nucleotides of the nucleotide sequence of SEQ ID NO: 2, 5, or 8 or of a nucleotide sequence having at least 60% identity, preferably at least 70% or at least 80%, more preferably at least 90%, at least 92%, at least 94%, at least 95%, at least 96%, at least 97%, at least 98% or at least 99% to the sequence of SEQ ID NO: 2, 5, or 8; or
C. a double-stranded RNA, wherein one strand corresponds to:
   a. at least 19 successive nucleotides of the nucleotide sequence of SEQ ID NO: 2, 5, or 8 or of a nucleotide sequence having at least 60% identity, preferably at least 70% or at least 80%, more preferably at least 90%, at least 92%, at least 94%, at least 95%, at least 96%, at least 97%, at least 98% or at least 99% to the sequence of SEQ ID NO: 2, 5, or 8, wherein T is replaced by U; or
   b. a nucleotide sequence which is complementary to at least 19 successive nucleotides of the nucleotide sequence of SEQ ID NO: 2, 5, or 8 or of a nucleotide sequence having at least 60% identity, preferably at least 70% or at least 80%, more preferably at least 90%, at least 92%, at least 94%, at least 95%, at least 96%, at least 97%, at least 98% or at least 99% to the sequence of SEQ ID NO: 2, 5, or 8, wherein T is replaced by U; or
D. an RNA-specific CRISPR/Cas system, such as a CRISPR/Cas13a system, directed against or targeting a nucleotide sequence encoding the F35H protein, or one or more polynucleotide sequence(s) encoding (and expressing or being capable of expressing) said RNA-specific CRISPR/Cas system; or
E. a chemical compound or an antibody altering (or being capable to alter) the enzymatic activity of the F35H protein upon interaction with said F35H, preferably reducing (or being capable to reduce) the enzymatic activity of the F35H protein or inhibiting (or being capable to inhibit) the F35H protein upon interaction with said F35H or increasing (or being capable to increase) the F35H protein upon interaction with said F35H.

In certain embodiments, the plant is not a plant variety.

In an aspect, the invention relates to a method for obtaining or generating or producing a plant or plant part, such as a maize or sorghum plant or sugar cane or plant part, preferably a maize plant or plant part, comprising (a) providing a first plant having a QTL allele, such as a QTL allele associated with improved digestibility as described herein elsewhere, optionally wherein said QTL allele is located on a chromosomal interval, preferably on chromosome 9, comprising and flanked by (molecular) marker alleles ma61070s01 and ma30168s02, preferably by marker alleles ma50827s01 and ma16983s02, more preferably by marker alleles ma17117s01 and ma61125s01, (b) crossing said first plant with a second plant, such as a second plant not having said QTL allele, (c) selecting progeny plants having said QTL allele, and optionally (d) harvesting said plant part from said progeny.

In certain embodiments, the QTL allele comprises one or more of the marker alleles of the invention as described herein elsewhere. In certain embodiments, the QTL allele comprises a mutated F35H gene as described herein elsewhere.

In an aspect, the invention relates to a method for obtaining or generating or producing a plant or plant part, such as a maize or sorghum or sugar cane plant or plant part, preferably a maize plant or plant part, comprising (a) providing a first plant having a (molecular) marker allele, such as a (molecular) marker allele associated with improved digestibility as described herein elsewhere, optionally wherein said (molecular) marker allele is located on a chromosomal interval, preferably on chromosome 9, comprising and flanked by (molecular) marker alleles ma61070s01 and ma30168s02, preferably by marker alleles ma50827s01 and ma16983s02, more preferably by marker alleles ma17117s01 and ma61125s01; (b) crossing said first plant with a second plant, such as a second plant not having said (molecular) marker allele, (c) selecting progeny plants having said (molecular) marker allele, and optionally (d) harvesting said plant part from said progeny.

In an aspect, the invention relates to a method for obtaining or generating or producing a plant or plant part, such as a maize or sorghum or sugar cane plant or plant part, comprising (a) providing a first plant having a mutated F35H gene or a first plant in which mRNA and/or protein expression of a F35H gene is altered, such as described herein elsewhere, preferably reduced or (substantially) eliminated or absent, such as described herein elsewhere, optionally wherein said mutated F35H gene is located on a chromosomal interval, preferably on chromosome 9, comprising and flanked by (molecular) marker alleles ma61070s01 and ma30168s02, preferably by marker alleles ma50827s01 and ma16983s02, more preferably by marker alleles ma17117s01 and ma61125s01, (b) crossing said first plant with a second plant, such as a second plant not having said mutated F35H gene, (c) selecting progeny plants having said mutated F35H gene, and optionally (d) harvesting said plant part from said progeny.

In certain embodiments, the QTL allele, marker allele, and/or F35H mutation in the first plant is present in a homozygous state. In certain embodiments the QTL allele, marker allele, and/or F35H mutation in the first plant is present in a heterozygous state. In certain embodiments, the QTL allele, marker allele, and/or F35H mutation in the second plant is present in a heterozygous state. In certain embodiments the QTL allele, marker allele, and/or F35H mutation in the second plant is not present.

In certain embodiments, the progeny is selected in which the QTL allele, marker allele, and/or mutated F35H is present in a homozygous state. In certain embodiments, the progeny is selected in which the QTL allele, marker allele, and/or mutated F35H is present in a heterozygous state.

In certain embodiments, the plant is or plant part is from maize.

In certain embodiments, the plant is or the plant part is from sorghum.

In certain embodiments, the plant is or the plant part is from sugar cane.

In certain embodiments, the methods for obtaining plants or plant parts as described herein according to the invention, such as the methods for obtaining plants or plant parts having improved digestibility, involve or comprise transgenesis and/or gene editing and/or base editing, such as including CRISPR/Cas, TALEN, ZFN, meganucleases; (induced) mutagenesis, which may or may not be random mutagenesis, such as TILLING. In certain embodiments, the methods for obtaining plants or plant parts as described herein according to the invention, such as the methods for obtaining plants or plant parts having improved digestibility, involve or comprise RNAi applications, which may or may not be, comprise, or involve transgenic applications. By means of example, non-transgenic applications may for instance involve applying RNAi components such as double stranded siRNAs to plants or plant surfaces, such as for instance as a spray. Stable integration into the plant genome is not required.

In certain embodiments, the methods for obtaining plants or plant parts as described herein according to the invention, such as the methods for obtaining plants or plant parts having improved digestibility, do not involve or comprise transgenesis, gene editing, base editing and/or mutagenesis.

In certain embodiments, the methods for obtaining plants or plant parts as described herein according to the invention, such as the methods for obtaining plants or plant parts having improved digestibility, involve, comprise or consist of breeding and selection.

In certain embodiments, the methods for obtaining plants or plant parts as described herein according to the invention, such as the methods for obtaining plants or plant parts having improved digestibility, do not involve, comprise or consist of breeding and selection.

In an aspect, the invention relates to a plant or plant part obtained or obtainable by the methods of the invention as described herein, such as the methods for obtaining plants or plant parts having improved digestibility.

In certain embodiments, the wild type or unmutated F35H gene comprises
(i) a nucleotide sequence comprising the sequence of SEQ ID NO: 1, 4, or 7;
   (ii) a nucleotide sequence having the cDNA or coding sequence of SEQ ID NO: 2, 5, or 8;
   (iii) a nucleotide sequence encoding for a polypeptide having the amino acid sequence of SEQ ID NO: 3, 6, or 9;
   (iv) a nucleotide sequence having at least 60% identity to the sequence of SEQ ID NO: 1, 2, 4, 5, 7, or 8; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity;
   (v) a nucleotide sequence encoding for a polypeptide having at least 60% identity to the sequence of SEQ ID NO: 3, 6, or 9; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity;
   (vi) a nucleotide sequence hybridizing with the reverse complement of a nucleotide sequence as defined in (i), (ii) or (iii) under stringent hybridization conditions; and
   (vii) a nucleotide sequence encoding a protein derived from the polypeptide encoded by the nucleotide sequence of any of (i) to (vi) by way of substitution, deletion and/or addition of one or more amino acid(s).

In certain embodiments, the wild type or unmutated F35H gene comprises
(i) a nucleotide sequence comprising the sequence of SEQ ID NO: 1, 4, or 7;
(ii) a nucleotide sequence having the cDNA or coding sequence of SEQ ID NO: 2, 5, or 8;
(iii) a nucleotide sequence encoding for a polypeptide having the amino acid sequence of SEQ ID NO: 3, 6, or 9;

In certain embodiments, the wild type or unmutated F35H gene comprises
(i) a nucleotide sequence having at least 60% identity to the sequence of SEQ ID NO: 1, 2, 4, 5, 7, or 8; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity; or
(ii) a nucleotide sequence encoding for a polypeptide having at least 60% identity to the sequence of SEQ ID NO: 3, 6, or 9; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity.

In certain embodiments, the wild type or unmutated F35H gene comprises
(i) a nucleotide sequence having at least 60% identity to the sequence of SEQ ID NO: 2, 5, or 8; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity; or
(ii) a nucleotide sequence encoding for a polypeptide having at least 60% identity to the sequence of SEQ ID NO: 3, 6, or 9; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity.

In certain embodiments, the wild type or unmutated F35H gene comprises
(i) a nucleotide sequence comprising the sequence of SEQ ID NO: 1, 4, or 7;
(ii) a nucleotide sequence having the cDNA or coding sequence of SEQ ID NO: 2, 5, or 8;
(iii) a nucleotide sequence encoding for a polypeptide having the amino acid sequence of SEQ ID NO: 3, 6, or 9;
(iv) a nucleotide sequence having at least 60% identity to the sequence of SEQ ID NO: 1, 2, 4, 5, 7, or 8 or SEQ ID NO: 2, 5, or 8; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity;
(v) a nucleotide sequence encoding for a polypeptide having at least 60% identity to the sequence of SEQ ID NO: 3, 6, or 9; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity;

In certain embodiments, the wild type or unmutated F35H gene comprises (i) a nucleotide sequence hybridizing with the reverse complement of a nucleotide sequence of SEQ ID NO: 1, 4, or 7 or SEQ ID NO: 2, 5, or 8, under stringent hybridization conditions.

In certain embodiments, the wild type or unmutated F35H gene comprises
(i) a nucleotide sequence encoding a protein derived from the polypeptide encoded by the nucleotide sequence of SEQ ID NO: 1, 4, or 7 or SEQ ID NO: 2, 5, or 8 by way of substitution, deletion and/or addition of one or more amino acid(s).

The skilled person will understand that the wild type or unmutated F35H gene product is a functional gene product having enzymatic activity, as defined herein elsewhere. The skilled person will further understand that sequence variations described above for the wild type F35H do not include frame shift or nonsense mutations.

As used herein, the mutated F35H or the mutation in the F35H may comprise or may refer to any type of F35H mutation. In certain embodiments the mutation alters expression of the wild type or native F35H protein and/or mRNA. In certain embodiments the mutation reduces or eliminates expression of the (wild type or native) F35H protein and/or mRNA, as described herein elsewhere. Mutations may affect transcription and/or translation. Mutations may occur in exons or introns. Mutations may occur in regulatory elements, such as promotors, enhancers, terminators, insulators, etc. Mutations may occur in coding sequences. Mutations may occur in splicing signal sites, such as splice donor or splice acceptor sites. Mutations may be frame shift mutations. Mutations may be nonsense mutations. Mutations may be insertion or deletion of one or more nucleotides. Mutations may be non-conservative mutations (in which one or more wild type amino acids are replaced with one or more non-wild type amino acids). Mutations may affect or alter the function of the F35H protein, such as enzymatic activity. Mutations may reduce or (substantially) eliminate the function of the F35H protein, such as enzymatic activity. Reduced function, such as reduced enzymatic activity, may refer to a reduction of about at least 10%, preferably at least 30%, more preferably at least 50%, such as at least 20%, 40%, 60%, 80% or more, such as at least 85%, at least 90%, at least 95%, or more. (Substantially) eliminated function, such as (substantially) eliminated enzymatic activity, may refer to a reduction of at least 80%, preferably at least 90%, more preferably at least 95%. Mutations may be dominant negative mutations. In certain embodiments, mutations are evaluated with reference to maize inbred line PH207, as defined herein elsewhere.

In certain embodiments, the F35H mutation is an insertion of one or more nucleotides in the coding sequence. In certain embodiments, the F35H mutation is a nonsense mutation. In certain embodiments, the F35H mutation results in altered expression of the F35H gene. In certain embodiments, the F35H mutation results in knockout of the F35H gene or knockdown of the F35H mRNA and/or protein. In certain embodiments, the mutation results in a frame shift of the coding sequence of F35H. In certain embodiments, the mutation results in an altered protein sequence encoded by the F35H gene.

In certain embodiments, the F35H mutation is an insertion, preferably in an exon, preferably an insertion in the first exon, of one or more nucleotides, preferably a frame shift insertion. In certain embodiments, the insertion is 187 nucleotides or about 187 nucleotides. In certain embodiments, the insertion is between position 97 and 98 of the F35H gene represented by the nucleotide sequence of SEQ ID NO: 1. The skilled person is capable of determining the corresponding position in F35H homologues or orthologues.

In certain embodiments, the insertion comprises or consists of the nucleotide sequence of SEQ ID NO: 10. In certain embodiments, the mutated F35H comprises the nucleotide sequence of SEQ ID NO: 11. Alternatively, the mutation is a substitution, preferably a substitution of at least one nucleic acid resulting in an exchange of at least one amino acid or resulting in the change of an amino acid coding codon into a stop codon. In preferred embodiments, the mutated F35H comprises the nucleotide sequence encoding one of the amino acid sequences selected from the group consisting of SEQ ID NO: 20, 22, 24, 26, 28, 30, 32, 34, 36, 38 or 40. Such nucleotide sequence may be selected from the group consisting of SEQ ID NO: 19, 21, 23, 25, 27, 29, 31, 33, 35, 37 or 39. More specific sequence information can be found in Table 3.

F35H mRNA and/or protein expression may be reduced or eliminated by mutating the F35H gene itself (including coding, non-coding, and regulatory element). Methods for introducing mutations are described herein elsewhere. Alternatively, F35H mRNA and/or protein expression may be reduced or eliminated by (specifically) interfering with transcription and/or translation, such as to decrease or eliminate mRNA and/or protein transcription or translation. Alternatively, F35H mRNA and/or protein expression may be reduced or eliminated by (specifically) interfering with mRNA and/or protein stability, such as to reduce mRNA and/or protein stability. By means of example, mRNA (stability) may be reduced by means of RNAi, as described herein elsewhere. Also miRNA can be used to affect mRNA (stability). In certain embodiments, a reduced F35H expression which is achieved by reducing mRNA or protein stability is also encompassed by the term “mutated” F35H. In certain embodiments, a reduced F35H expression which is achieved by reducing mRNA or protein stability is not encompassed by the term “mutated” F35H.

In certain embodiments, the (molecular) marker alleles which are associated with improved digestibility as described herein are defined as follows:
ma61134xxx is an insertion of one or more nucleotides between position 134254381 and 134254382 of chromosome 9 referenced to line PH207, preferably an insertion as set forth in SEQ ID NO: 12; and/or
ma61070s01 is a single nucleotide polymorphism (SNP) at position 121588825 of chromosome 9 referenced to line PH207, wherein said nucleotide is A or T, preferably a single nucleotide polymorphism (SNP) as set forth in SEQ ID NO: 13; and/or
ma30168s02 is a single nucleotide polymorphism (SNP) at position 139452428 of chromosome 9 referenced to line PH207, wherein said nucleotide is A or G, preferably a single nucleotide polymorphism (SNP) as set forth in SEQ ID NO: 14; and/or
ma50827s01 is a single nucleotide polymorphism (SNP) at position 127454426 of chromosome 9 referenced to line PH207, wherein said nucleotide is A or G, preferably a single nucleotide polymorphism (SNP) as set forth in SEQ ID NO: 15; and/or
ma16983s02 is a single nucleotide polymorphism (SNP) at position 137363784 of chromosome 9 referenced to line PH207, wherein said nucleotide is A or G, preferably a single nucleotide polymorphism (SNP) as set forth in SEQ ID NO: 16; and/or
ma17117s01 is a single nucleotide polymorphism (SNP) at position 132038900 of chromosome 9 referenced to line PH207, wherein said nucleotide is A or G, preferably a single nucleotide polymorphism (SNP) as set forth in SEQ ID NO: 17; and/or
ma61125s01 is a single nucleotide polymorphism (SNP) at position 135947973 of chromosome 9 referenced to line PH207, wherein said nucleotide is A or G, preferably a single nucleotide polymorphism (SNP) as set forth in SEQ ID NO: 18;
preferably wherein PH207 refers to Zea mays inbred line as described in “Draft Assembly of Elite Inbred Line PH207 Provides Insights into Genomic and Transcriptome Diversity in Maize”, Hirsch et al., Plant Cell. 2016 November; 28 (11): 2700-2714. Published online 2016 Nov. 1. doi: 10.1105/tpc.16.00353.

In certain embodiments, the insertion associated with marker allele ma61134xxx is a frame shift insertion. In certain embodiments, the insertion associated with marker allele ma61134xxx is an insertion of the nucleotide sequence of SEQ ID NO: 10. In certain embodiments, marker allele ma61134xxx comprises or consists of the (contiguous) nucleotide sequence of SEQ ID NO: 12.

In certain embodiments, marker allele ma61070s01 comprises or consists of the (contiguous) nucleotide sequence of SEQ ID NO: 13.

In certain embodiments, marker allele ma30168s02 comprises or consists of the (contiguous) nucleotide sequence of SEQ ID NO: 14.

In certain embodiments, marker allele ma50827s01 comprises or consists of the (contiguous) nucleotide sequence of SEQ ID NO: 15.

In certain embodiments, marker allele ma16983s02 comprises or consists of the (contiguous) nucleotide sequence of SEQ ID NO: 16.

In certain embodiments, marker allele ma17117s01 comprises or consists of the (contiguous) nucleotide sequence of SEQ ID NO: 17.

In certain embodiments, marker allele 61125s01 comprises or consists of the (contiguous) nucleotide sequence of SEQ ID NO: 18.

In an aspect, the invention relates to the use of one or more of the (molecular) markers described herein for identifying a plant or plant part having improved digestibility. In an aspect, the invention relates to the use of one or more of the (molecular) markers described herein which are able to detect at least one diagnostic marker allele for identifying a plant or plant part having improved digestibility. In an aspect, the invention relates to the detection of one or more of the (molecular) marker alleles described herein for identifying a plant or plant part having improved digestibility.

The marker alleles of the invention as described herein may be diagnostic marker alleles which are useable for identifying or selecting plants or plant parts having improved digestibility, preferably improved stover digestibility.

In an aspect, the invention relates to a (isolated) polynucleic acid comprising a (molecular) marker allele of the invention, or the complement or the reverse complement of a (molecular) marker allele of the invention. In certain embodiments, the invention relates to a polynucleic acid comprising at least 10 contiguous nucleotides, preferably at least 15 contiguous nucleotides or at least 20 contiguous nucleotides of a (molecular) marker allele of the invention, or the complement or the reverse complement of a (molecular) marker allele of the invention. In certain embodiments, the invention relates to a polynucleic acid comprising at least 10 contiguous nucleotides, preferably at least 15 contiguous nucleotides or at least 20 contiguous nucleotides of any of SEQ ID NOs: 10, 12, 13, 14, 15, 16, 17, or 18, or the complement or the reverse complement of any of SEQ ID NOs: 10, 12, 13, 14, 15, 16, 17, or 18. In certain embodiments, the polynucleic acid is capable of discriminating between a (molecular) marker allele of the invention and a non-molecular marker allele, such as to specifically hybridise with a (molecular) marker allele of the invention. In certain embodiments, the polynucleic acid is capable of hybridising with a unique nucleotide fragment or section of any of SEQ ID NOs: 10, 12, 13, 14, 15, 16, 17, or 18, or the complement or the reverse complement of any of SEQ ID NOs: 10, 12, 13, 14, 15, 16, 17, or 18. It will be understood that a unique section or fragment preferably refers to a section or fragment comprising the SNP or the respective marker alleles of the invention (such as marker alleles ma61070s01, ma30168s02, ma50827s01, ma16983s02, ma17117s01 or ma61125s01), or a section or fragment comprising the 5′ or 3′ junction of the insert of a marker allele of the invention or a section or fraction comprised within the insert of a marker allele of the invention (such as marker allele ma61134xxx). In certain embodiments, the polynucleic acid or the complement or reverse complement thereof does not (substantially) hybridise with or bind to (genomic) DNA originating from maize inbred line PH207. In certain embodiments, the sequence of the polynucleic acid or the complement or reverse complement thereof does not occur or is not present in maize inbred line PH207.

In an aspect, the invention relates to a polynucleic acid capable of specifically hybridizing with a (molecular) marker allele of the invention, or the complement thereof, or the reverse complement thereof.

In certain embodiments, the invention relates to a polynucleic acid specifically hybridising with any of the sequences of SEQ ID NOs: 10, 12, 13, 14, 15, 16, 17, or 18, or the complement or the reverse complement thereof.

In certain embodiments, the polynucleic acid is a primer. In certain embodiments, the polynucleic acid is a probe.

In certain embodiments, the polynucleic acid is an allele specific polynucleic acid, such as an allele specific primer or probe.

In certain embodiments, the polynucleic acid comprises at least 15 nucleotides, such as 16, 17, 18, 19, 20, 21, 22, 23, 24, or 25 nucleotides, such as at least 30, 35, 40, 45, or 50 nucleotides, such as at least 100, 200, 300, or 500 nucleotides.

It will be understood that “specifically hybridizing” means that the polynucleic acid hybridises with the (molecular) marker allele (such as under stringent hybridisation conditions, as defined herein elsewhere), but does not (substantially) hybridise with a polynucleic acid not comprising the marker allele or is (substantially) incapable of being used as a PCR primer. By means of example, in a suitable readout, the hybridization signal with the marker allele or PCR amplification of the marker allele is at least 5 times, preferably at least 10 times stronger or more than the hybridisation signal with a non-marker allele, or any other sequence.

In an aspect, the invention relates to a kit comprising such polynucleic acids, such as primers (comprising forward and/or reverse primers) and/or probes. The kit may further comprise instructions for use.

In will be understood that in embodiments relating to a set of forward and reverse primers, only one of both primers (forward or reverse) may need to be capable of discriminating between a (molecular) marker allele of the invention and a non-marker allele, and hence may be unique. The other primer may or may not be capable of discriminating between a (molecular) marker allele of the invention and a non-marker allele, and hence may be unique.

In a further aspect, the invention relates to a method for producing an ensilaged plant material or animal feed having improved digestibility, comprising (a) growing the plant according to the present invention, (b) harvesting the plant or plant parts, (c) optionally, chopping and/or crushing the plant, and (c) ensiling the plant, optionally by adding a stimulant like a bacterial inoculant, a sugar, and an enzyme. Furthermore, the invention relates to an ensilaged plant material or animal feed produced by said method.

In an aspect, the invention relates to a method for producing biogas or bioethanol, comprising the following steps: (a) providing the plant or plant parts according to the present invention or the ensilaged plant material according to the present invention, and (b) producing biogas or bioethanol from the plant or the ensilaged plant material.

The aspects and embodiments of the invention are further supported by the following non-limiting examples.

### EXAMPLES

### Example 1

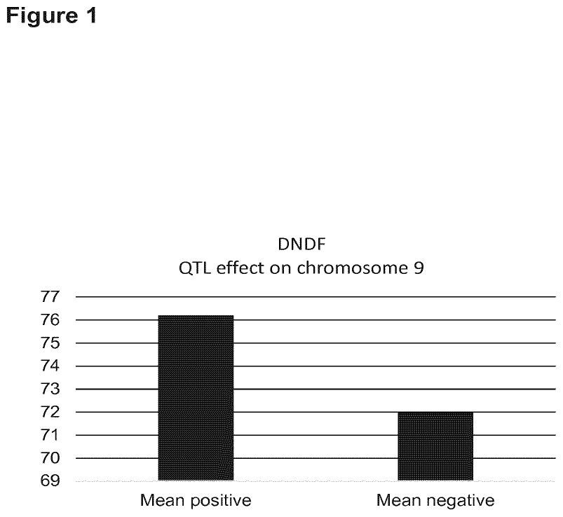

A QTL experiment on stover digestibility was carried out in two DH (double haploid) populations. In both populations, a QTL with strong effect was identified on the same chromosomal position on chromosome 9 (FIG. 1). The QTL region seems to not contain any of known characterized genes of the lignin metabolism.

Marker analysis using high density SNP genotyping with a SNP array showed no polymorphisms between the line harbouring the positive allele of the QTL and other commercially available lines. Taking advantage of this effect, a sequence capture experiment was carried out using a QTL carrying line and a control line. As at that time, the only genomic reference sequence was AGPv02, probes for sequence capture were developed on this reference. Data analysis turned out to be very difficult due to high repetitiveness of the region and partly low similarity between the region in B73 (AGPv02) and the QTL allele. Out of this experiment one marker, ma60405s01, was developed showing a polymorphism between the two lines in the QTL region.

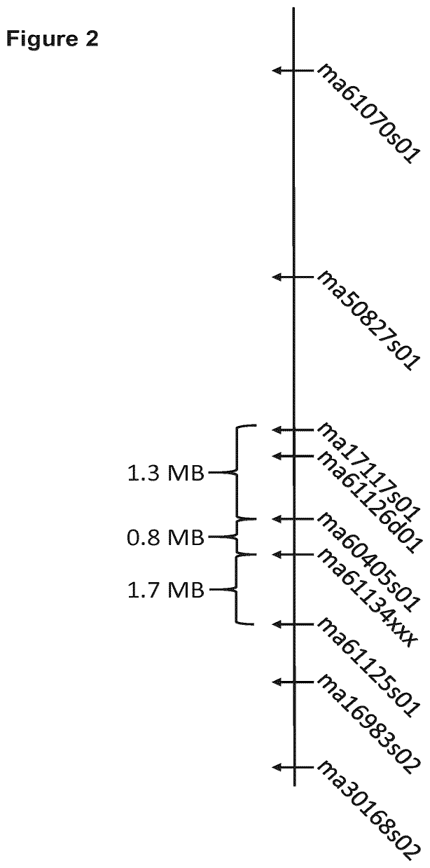

In order to identify further preferably polymorphic markers, a WGS (Whole Genome Sequencing) of one QTL carrying line was performed. Scaffolds covering the target were selected and compared to the reference genome of PH207 (“Draft Assembly of Elite Inbred Line PH207 Provides Insights into Genomic and Transcriptome Diversity in Maize”, Hirsch et al., Plant Cell. 2016 November; 28 (11): 2700-2714. Published online 2016 Nov. 1. doi: 10.1105/tpc.16.00353). Three more polymorphic markers could be identified (ma61126d01, ma61134xxx and ma61125s01, see FIG. 2). Their QTL specific alleles are unique and cannot be detected in any KWS line used for silage maize breeding.

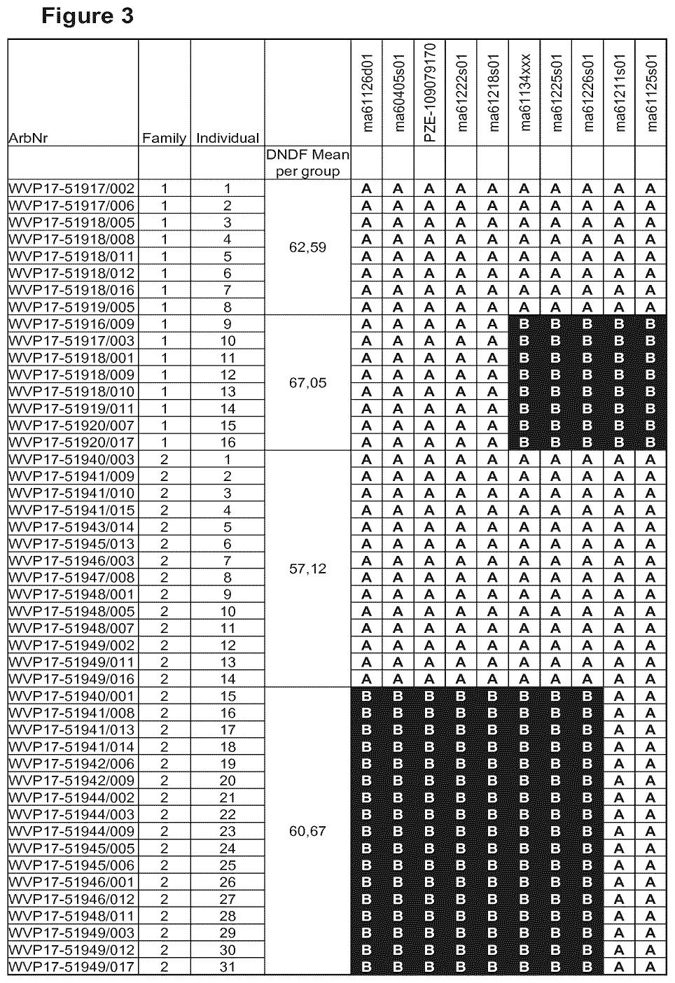

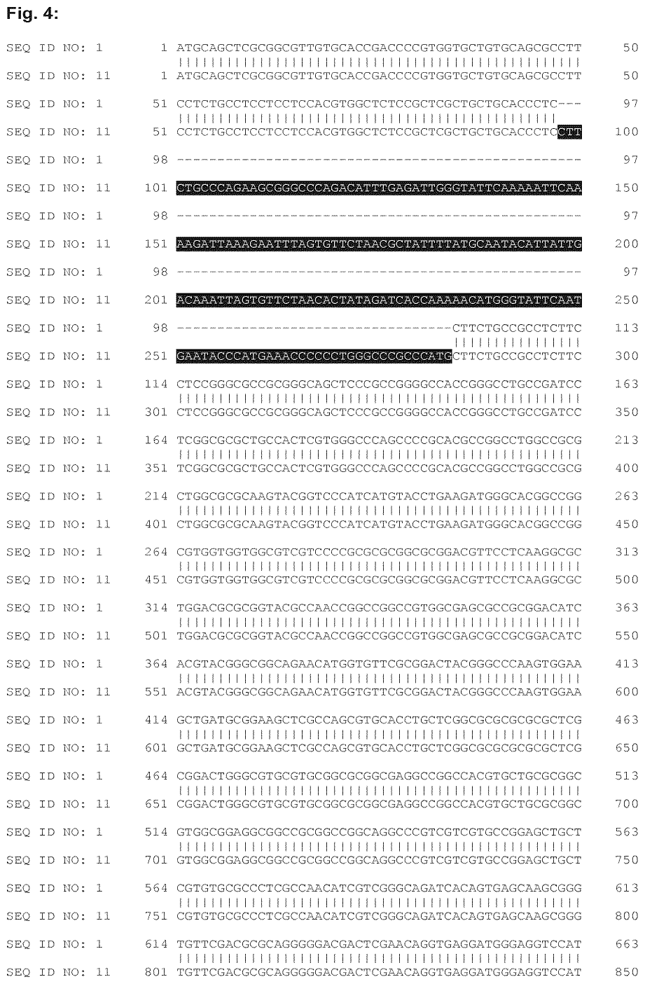

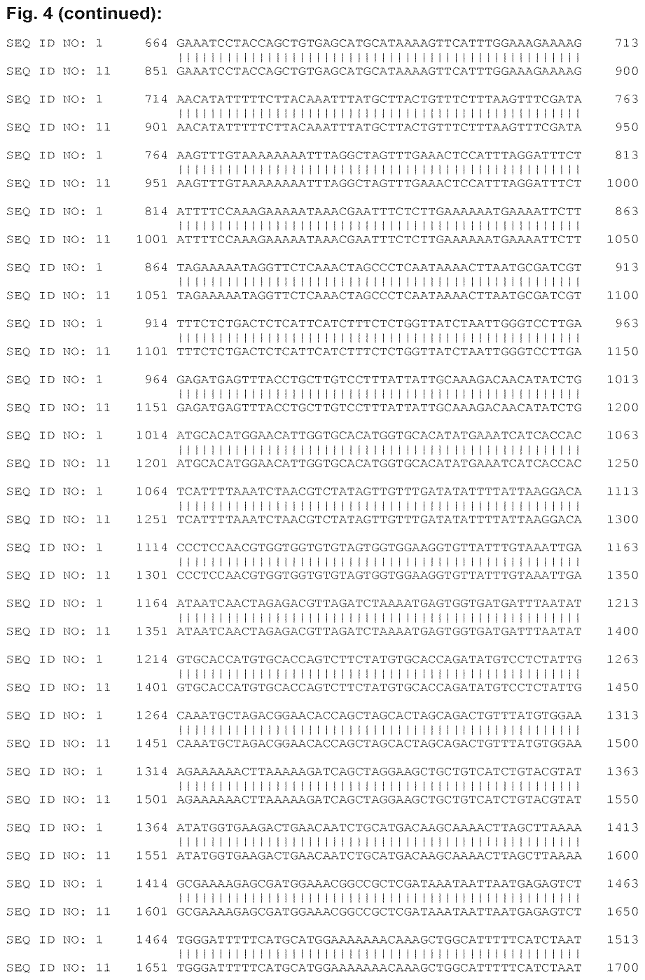

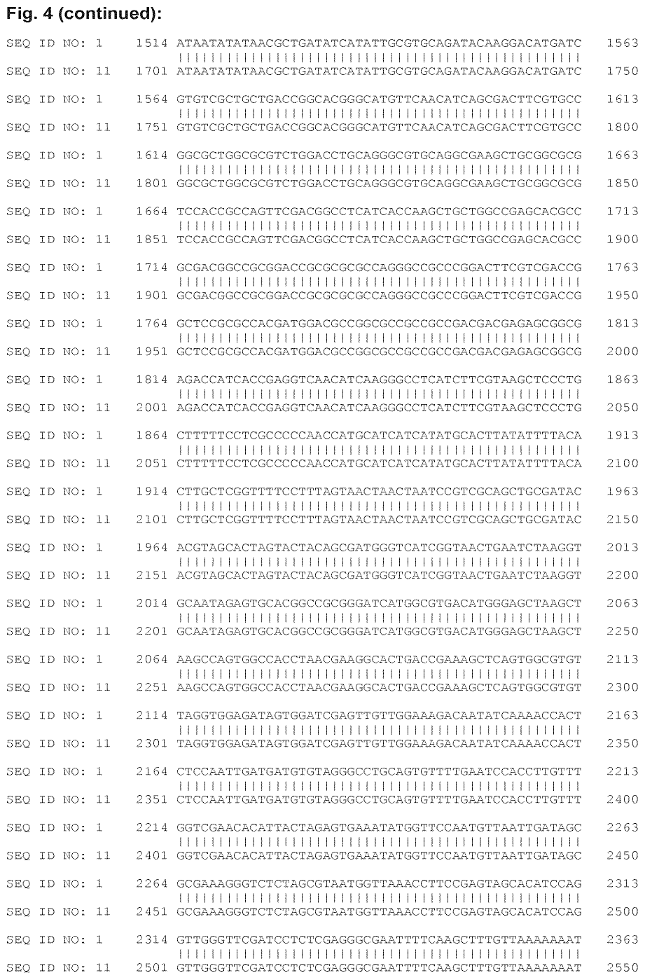

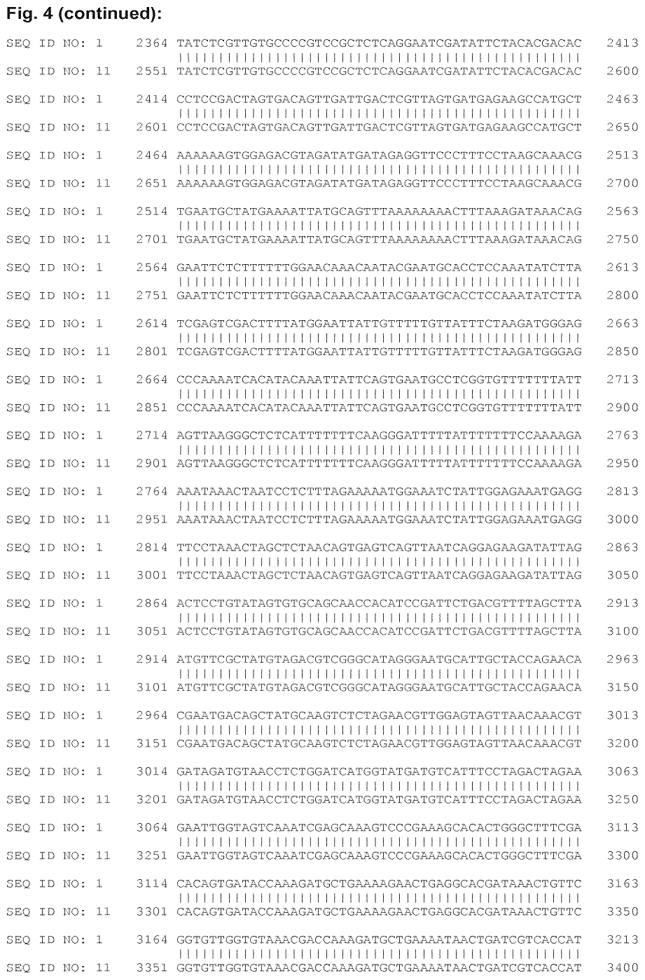

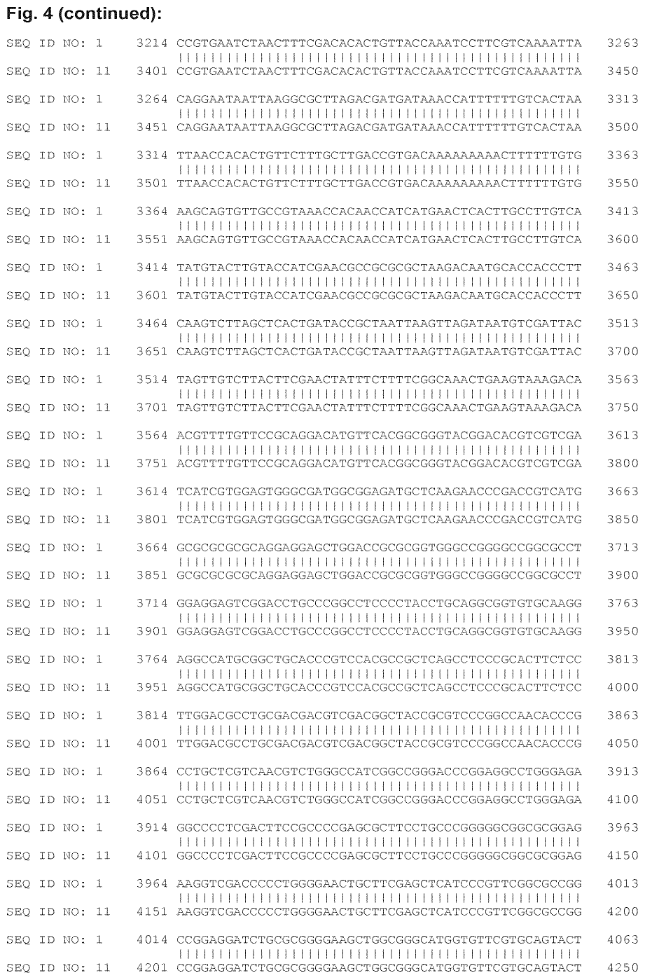

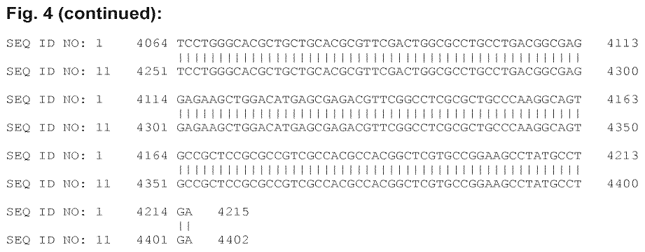

Fine-mapping of the QTL has narrow down the region first to 16.7 MB and in the last step to approx. 719kb on the PH207 reference (see FIG. 3 and Table 1). FIG. 3 shows two families of recombinants derived of the QTL line (B) crosses with a line not carrying the QTL (A). The indicated DNDF is the mean of all family members with or without QTL respectively. The marker, which is best associated with the phenotype in the latest recombinants, is ma61134xxx. It represents an insertion of 187 bp in a gene coding for a cytochrome P450 flavonoid 3′,5′-hydroxylase (called F35H, see FIG. 4). This insertion causes an elongation of the N-terminus of the protein and an early stop codon leading likely to a knockout of the gene. The corresponding gene of AGPv02 is expressed in leaves and stem. It stands at the beginning of the flavonoid metabolism taking resources from the lignin metabolic pathway.

TABLE 1
List of markers for silage QTL or the gene as such.

| | | genetic map | PH207-public |
|---|---|---|---|
| marker | Chrom | [position in cM] | [position in bp] |
| ma61070s01 | 9 | | 121588825 |
| ma50827s01 | 9 | | 127454426 |
| ma17117s01 | 9 | | 132038900 |
| ma60405s01 | 9 | 55, 054 | 133444836 |
| ma61134xxx | 9 | 55, 98 | 134254381 |
| ma61125s01 | 9 | 56, 224 | 135947973 |
| ma16983s02 | 9 | | 137363784 |
| ma30168s02 | 9 | | 139452428 |

Within the region of 133,4-135,9MB of chromosome 9 in PH207 all other hypothetical genes derived of internal maker annotation of PH207 were checked for polymorphism between the QTL line and PH207. Out of more than 100 genes, only the described one showed a polymorphism. 79 genes were identical. The rest was mostly repetitive or only partly represented in the assembly of the QTL-line.

The capillary marker ma61134xxx could be converted to a pair of dominant KASP markers and to a codominant KASP marker, all three are available for routine use.

Most important diagnostic marker ma61134xxx is directed to the insertion in the causative gene. The insertion is present in the genotype carrying the silage QTL and absent in reference line PH207. In PH207 the following sequence is not present in the gene (SEQ ID NO: 10):

CTTCTGCCCAGAAGCGGGCCCAGACATTTGAGATTGGGTATTCAAAAAT
TCAAAAGATTAAAGAATTTAGTGTTCTAACGCTATTTTATGCAATACAT
TATTGACAAATTAGTGTTCTAACACTATAGATCACCAAAAACATGGGTA
TTCAATGAATACCCATGAAACCCCCCTGGGCCCGCCCATG

The person skilled in the art is able to design markers for known marker systems which allows the detection of the presence or absence of the insertion.

Furthermore, one skilled in the art is also able to find markers for known marker systems which are suitable for further analysing of the silage QTL region as well as markers with diagnostic value basing for instances on single nucleotide polymorphisms (SNPs) or InDels (see exemplary Table 2).

TABLE 2
Marker alleles in QTL line

| Marker | Allele of QTL line |
|---|---|
| ma61070s01 | T (comprised in SEQ ID NO: 13) |
| ma50827s01 | A (comprised in SEQ ID NO: 15) |
| ma17117s01 | A (comprised in SEQ ID NO: 17) |
| ma61134xxx | cttctgcccagaagcgggcccagacatttgagattgggtattcaaa |
| | aattcaaaagattaaagaatttagtgttctaacgctattttatgca |
| | atacattattgacaaattagtgttctaacactatagatcaccaaaa |
| | acatgggtattcaatgaatacccatgaaacccccctgggcccgccc |
| | atg (SEQ ID NO: 10, comprised in SEQ ID NO: 12) |
| ma61125s01 | G (comprised in SEQ ID NO: 18) |
| ma16983s02 | G (comprised in SEQ ID NO: 16) |
| ma30168s02 | G (comprised in SEQ ID NO: 14) |

In conclusion, the present invention describes the identification of a marker haplotype spanning 3,8MB of PH207 (see FIG. 2) and describing the genotype of the QTL line in the target region: The set of markers identified during the mapping (see Table 1 and 2) can be used to integrate the positive QTL in any relevant genetic background. Most important is the marker diagnostic for the insertion which is the functional mutation. However, the use of markers outside of the gene in flanking region closely linked to the gene can also be used in order to identify the genotype of QTL line. The unique haplotype of markers (see FIG. 2) can be used for marker assisted applications, i.e. for trait introgression through backcrossing or forward breeding and for monitoring of the presence of the unique silage haplotype. In addition, the developed markers can be used to increase the genetic variation in this chromosomal region and keeping the advantageous silage allele. Further described is the identification of a gene for the target: The knowledge of the gene and the found insertion (functional mutation) can be used for increasing genetic variability in this locus either by tilling or by genome editing or genetic modification to further improve the effect.

### Example 2

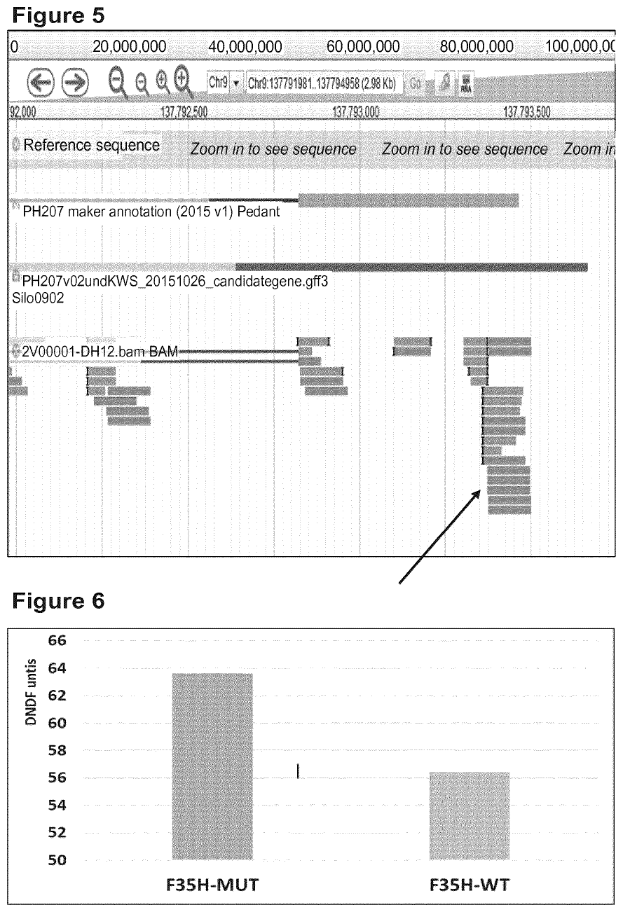

For functional validation RNAseq analyses on leaf material of two QTL lines carrying the mutated F35H in a dent and a flint background are conducted. Line 5F279 without mutated F35H serves as control. The results show that the insertion is expressed and causes a frameshift (FIG. 5).

Additionally, several TILLING mutants for the native F35H gene have been identified in an EMS Tilling population of PH207 and predicted (Table 3). Two TILLING mutants (P434L (called PH207m023a) and W426stop (called PH207m023b)) has been used for validation.

TABLE 3
Identified and predicted (*) TILLING mutations

| gene | mutant ID | mutation | gDNA | protein |
|---|---|---|---|---|
| F35H-MUT | PH207m023a | P434L | SEQ ID | SEQ ID |
| | | | NO: 19 | NO: 20 |
| F35H-MUT | PH207m023b | W426stop | SEQ ID | SEQ ID |
| | | | NO: 21 | NO: 22 |
| F35H-MUT | PH207m023c | R252W | SEQ ID | SEQ ID |
| | | | NO: 23 | NO: 24 |
| F35H-MUT | PH207m023d | R405H | SEQ ID | SEQ ID |
| | | | NO: 25 | NO: 26 |
| F35H-MUT | PH207m023e | P407L | SEQ ID | SEQ ID |
| | | | NO: 27 | NO: 28 |
| F35H-MUT | PH207m023f | E427K | SEQ ID | SEQ ID |
| | | | NO: 29 | NO: 30 |
| F35H-MUT | PH207m023g | G450R | SEQ ID | SEQ ID |
| | | | NO: 31 | NO: 32 |
| F35H-MUT | PH207m023h* | P429S | SEQ ID | SEQ ID |
| | | | NO: 33 | NO: 34 |
| F35H-MUT | PH207m023i* | P429L | SEQ ID | SEQ ID |
| | | | NO: 35 | NO: 36 |
| F35H-MUT | PH207m023j* | R436C | SEQ ID | SEQ ID |
| | | | NO: 37 | NO: 38 |
| F35H-MUT | PH207m023k* | Q44stop | SEQ ID | SEQ ID |
| | | | NO: 39 | NO: 40 |

In pre-trial it has been showed already that the identified QTL effect can be rapidly screened under greenhouse conditions. In a first generation, seeds from the new identified F35H mutants has been grown to plants, selfed and homozygous wildtype and homozygous mutant plants have been selected. Every homozygous class represents one class. The classes are distinguished by different fixation of background mutations. From these selected homozygous seeds plants have been grown for phenotyping in a second generation. Thereby, different mutant classes could be tested and phenotyped. All classes were tested for DNDF (Digestible Neutral Detergent Fiber; Table 4) and the average for wildtype and mutant classes was calculated. For the wildtype group, the average was 56,7 and for the mutant group, it was 63,7, which is significantly higher.

TABLE 4
Greenhouse trial for fast validation of Mutants
for the F35H gene

| gene | mutant ID | repetitions | versions | PDNDF |
|---|---|---|---|---|
| F35H-MUT | PH207m023a | 1 | 20 | 63.7 |
| F35H-WT | PH207m023a | 1 | 20 | 56.4 |
| F35H-MUT | PH207m023b | 4 | 2 | 64.1 |
| F35H-WT | PH207m023b | 2 | 1 | 62.9 |

Mutant lines showed the same or even improved effects with respect to digestibility. Thus, the newly identified mutations in F35H represents allelic variants which improves digestibility significantly. In particular mutant PH207m023a showed a strong effect (FIG. 6).

1-15. (canceled)

16. A method for identifying a maize plant or plant part having improved digestibility comprising:
a) screening for the presence of a QTL allele associated with improved digestibility, said QTL allele comprising a nucleotide sequence of a gene encoding a cytochrome P450 flavonoid 3′,5′-hydroxylase (F35H) located on chromosome 9, having a mutation leading to reduced or absent expression of the mRNA of the F35H gene and/or the F35H protein, or a mutation leading to an F35H protein having reduced enzymatic activity upon translation or a nonfunctional F35H protein, or
b) screening for reduced or absent expression of the mRNA of a gene encoding F35H and/or the F35H protein or for an F35H protein having reduced enzymatic activity upon translation or a nonfunctional F35H protein, or
c) screening for the presence of a mutation leading to reduced or absent expression of the mRNA of the F35H and/or the F35H protein, or a mutation leading to an F35H protein having reduced enzymatic activity upon translation or a nonfunctional F35H protein; and
selecting a maize plant or plant part having reduced or absent expression of the mRNA of the F35H and/or the F35H protein, or an F35H protein having reduced enzymatic activity upon translation or a nonfunctional F35H protein identified in steps a)-c),
wherein the unmutated F35H is selected from the group consisting of:
(i) a nucleotide sequence comprising the sequence of SEQ ID NO: 1;
(ii) a nucleotide sequence having the cDNA of SEQ ID NO: 2;
(iii) a nucleotide sequence encoding for an amino acid sequence having the amino acid sequence of SEQ ID NO: 3;
(iv) a nucleotide sequence having at least 90% identity to the sequence of SEQ ID NO: 1 or 2; and
(v) a nucleotide sequence encoding for a polypeptide having at least 90% identity to the sequence of SEQ ID NO: 3.

17. The method according to claim 16, the method further comprising screening for the presence of the molecular marker allele of ma61134xxx and/or one or more molecular marker alleles located in a chromosomal interval on chromosome 9 flanked by marker alleles ma61070s01 (SEQ ID NO: 13) and ma30168s02 (SEQ ID NO: 14), by marker alleles ma50827s01 (SEQ ID NO: 15) and ma16983s02 (SEQ ID NO: 16), or by marker alleles ma17117s01 (SEQ ID NO: 17) and ma61125s01 (SEQ ID NO: 18).

18. The method according to claim 16, wherein the QTL allele is flanked by marker alleles ma61070s01 (SEQ ID NO: 13) and ma30168s02 (SEQ ID NO: 14), or by marker alleles ma50827s01 (SEQ ID NO: 15) and ma16983s02 (SEQ ID NO: 16), or comprises and is flanked by marker alleles ma17117s01 (SEQ ID NO: 17) and ma61125s01 (SEQ ID NO: 18).

19. A plant or plant part having improved digestibility identified by the method according to claim 16.


SEQUENCE LISTING (40 sequences)

SEQ ID NO: 1 - DNA, 4215 bp, Zea mays
  source 1..4215: mol_type = unassigned DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 2 - DNA, 1584 bp, synthetic construct
  misc_feature 1..1584: note = cDNA of SEQ ID NO: 1
  source 1..1584: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 3 - protein, 527 aa, Zea mays
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWERPLDFRPERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 4 - DNA, 5903 bp, Sorghum bicolor
  source 1..5903: mol_type = unassigned DNA
cccctacgatgtatatgtagctcgtctgaaaacggttaaataaaaaaaaaagataacaacggtcccgagatttcgagtacacagcgcctcatcaccaatataaaaaccgtcgtgctcgcggaggcatccttagcttgggcaggtcctcctccctagctagctagcacagtaacgccaccatgaagctcgccgcgttgtgcaccgaccccgtcgtgctctcctgcgccttcctctgcctcctcctccacctcgctctccggtccctgctgcacccttcttctgccgcctcttcctctggccgccgcgccggcgccggcggccacctcccgccggggccaccgggcctgccgatcctcggcgcgctgccactcgtcggccctgccccgcatgccggcctggccgcgctggcgcgcaagtacggtcccatcatgtacctgaagatgggcacgacgggcgtcgtggtggcgtcgtccccgtgcgcggcgaagacgttcctcaaggcgctggacgcgaagtacgccaaccggccggcggtggcgagcgccgccgacatcacgtaccggcgccagaacatggtgttcgcggactacgggcccaagtggaagctgatgcggaagctcgccagcgtgcacctgctcggggcgcgcgcgctcgccgactgggcgtgcgtgcggcgcgacgaggccggccacctgctccgcggcgtggcggaggcggccgcggcccggcggcccgtcgtcgtgccggagctgctcgtgtgcgcgctcgccaacatcgtcgggcagatcaccgtcagcaagcgggtgttcgacgcgcagggggatgactcaaacaggtgaggatcgatcaaagctgcatgaaatcctatcaacaagacaatgaccatatatatataaggcataaaatccaagttcatttcgaaaggaaagcacatgtttttcttccaaaatttcatgattatcgggtgccttaagttgggatgaggtttacaatatctttgaatgggactagtgagatcagttgaactagttatccttaattaatgcaatgcttgacggaatgaacatcagcagaccgttaatgcggaaagccggggataaaaaaattaaaaagttcagctaggaagctgctgtcatcgggtacgtatgagaagactgaagaatcggcatgacgagcacttagtactggcatttagccggaatgtccttgttagtgcagagagtcgctcaccaaaacctgaaagcgaaaagagagcgatcgaaacggcccctccatcgacaaataataaggagaatcttgtcatttttcatgcatgcattcatggaaaaaacacaaactgatcaagtatttttcgtctaaacatataataattctgataccatatatgcgtgctgcgtgctgcgtgctgcgtgcaggtacaaggacatgatcgtgtcgctgctgacgggcacgggcatgttcaacatcagcgacttcgtgccggcgctggcgcgtctggacctgcagggcgtgcaggccaagctgcggcggatccaccaccagttcgacggcctcatcaccaagctgctggccgagcacgccgcgacggcggcggaccgcgcgcgccagggccgccaggacttcgtcgacaggctccgcgccacgatggacgccggcgccgacgacgagagcggcgagaccatcaccgaggtcaacatcaagggcctcatcttcgtaagctctcgctctctttatattttcctcacccccaacctctcttcatgcatttatattttaattgcttgcttcccttggttgctaactaaccaactactaatctgtcgtagctgcaaaaatatgtaatactactgcaacaatcgatcatggccatgggtaactgaatcatatactccctccatcccaaattgtaagtcattccaagaattttgaagagtcaaacttttcaaagtttgaccaaatttatatgataaaataataattattatgataccaactaaatatcattagattcttccttaattatattttcatagtatactcatcttacgttacaaatcttagtatttcactctataattttggtcaaacgtgaaaatgctttgactctccaagattcttggaatgacttacaatttgagatggagggagtatatcttcgctgaaaatgaagtgcacggcattttggtgtgacataggagctaagctaagctaagctagtagccaccggacctaacggagcactgacactgaccgaaagctcagcgtgttaggtgaagctgctagtggagttgttggaaagacaatatcaaaatcacaatctcgaactcatgagatgatgtggggcctgcagggccggttttgagtcctccatgatggtgatagctcaaggcttgtttggttcaaacattactagagcaaaatatggttcaaacattaattgctaaccgttgaagagtagaatgcatatatatgcctgtcatgccatggttttatgagaagatgcttactatatatttttattagataacaatagggatcactagatttaattatctgccgagagaacatacagatttaatccaacctaaattcacccccgtctccttgttctcacggtggaaatatatagtgggtaaactgacaaaaaaggcatttggttcctatactttgttggaacatatttttaatttgttaggctatatagcatacagatccaacaaattccacccataaaatatctatcaatacgcataaaagtagtttaaggaaatctagaaatattggaaccactactagctagaatctgtatgttttcctaggattttaattcctacgaatagaactaaaatatgtaaggaaaacatttccctatcatattttgcaccaaaaaaaattctatcgatgcataaatagtaaaacacgaaaaatgttgggacaatagggacttatttaccccaagggaaccaaagcacctaaaaattatacatttcctgaaggaaatttagatagtagttccaagaaaaactagaaaagtggtggcatagatatgctagggaattcattccctaagggaatgagaatgtatgaaaattatacaatttcaaataagctctaaagataaagaggtattctcttttctggaacaaacaaaatatgaaagtgcctccaaagatcttattgagtcaacttttatagaattatatattgtttttattatttttaagatggcgtgcaaaatcaagtctaaattattcagtgaaagcctcgagattctttaaggattaaaaataagtgaagcggttaagatttaatcaggaaaacgatattggactcctgtcctccaaccaaacttgattatgacgttttagcttaatgttcactttgtagacgtcggatatatagggatgcgaaccgaaaggaaaaggctgacgttttaacaaactgcacatatatgttaccagaactcgaatagcagctttgtaagtctctagaatgttggagtagttaacaacgtgatagacgatgtaacacctggaccatgttattatgtcatttcctagaagaattgtaatcaaatctattaattaatcaagcaaagaattgaaagctataaatggcttaaggcaacatctggttgtgagttgtgtgattaattattggtgaatgtcgtgtcagcactaacattattaaggtcagtctcaatgcatagtttcatgacacagttaccaagactataaactaggtaaccgagccacaagagtttcatggggatgaaactcctctctcatctgatgaaactccttcatttaatgactctgccaactcagcaattttgcttatgtggcaccatatttaatgtgcatgacactctcataaaacatgcattgagactggcctaagggggtgtttagtgttggactctttagatcgtctactaggtcgtagatcggtggggaatttacttctatttgggataaaccacatagaatttcatggatctagagaggaatggaggacgagaggatgtgttatatactgaaccatacctttggggttggcggagaagcgcgtggtcgaggcagtggcggtggcggactttccgtcgctggcagtgcctccctctcgatcggtctagggttagatggggactcgcggcattgtagcggtgaaccttgtaacttgtgctctagcctccaccctctcttttatacactgcgcgacaggggcctggcagccaagttattgggctagacgcccccgatcagggcgtggtcaagggtccggccaagtctttggacttgggccagtggagatcaatctaacatttagttccccataaaatacaaaatgtcaactactttggaatgatgaaatgcaaaatatcaaaaatttcaggttagatccatccaaaatgcagaatttattgctctcattatagcctcttgagacttttttggacttttttggcctcttgaggccaaaatacaaaatagagaataaccacatttttgttaaaatttttgcatagtatttatttttattatgtaaaataatacaccaaaactcagaattttttgggatggaaggggaactaaacaccccgtgaatctagtcggcacaatgactcaagcaatgaaaccactactgtttcagtataaggttggaaaacactgttctcaacataacatcgcattgactctctctttgtgggctgtgtggctttgtgggtcagcttgattggagctccttaacatatataacatacactgtgcctgctgatgctgaggttgtactgtcatcaaacacactaaaacagtgcaccacacccttcaataatgtcaggtcaaactatataaccagcttatattatatacagtgttaaacttaaacccaacccaacgatatataattcacttgcacatacttcaaaaccatgtatatatatctcgttgaaaaaaattataggagtaataatgtttgcgtgcgacgcatcgctgctttgcatgcacgcaggacatgttcacggcgggcacggacacgtcgtccatcatcgtggagtgggcgatggcggagatgctcaagaacccgtccgtgatggcgcgcgcgcaggaggagctggaccgcgtggtgggccgcggccgccgcctggaggagtcggacctgcccagcctcccctacctgcaggccgtctgcaaggaggccatgcggctgcacccgtcgacgccgctcagcctcccgcacttctccttcgacgcctgcgacgacgacgtcgccgccggcggctaccgcgtcccggccaacacccggctgctcatcaacatctgggccatcggccgggacccggcggcgtggaagaagcccctggagttccggcccgagcggttcctgccgggcggcggcgccgagaaggtcgacccgatggggaactgcttcgagctcatcccgttcggcgccggcaggaggatctgcgccgggaagctggccggcatggtgttcgtgcagtacttcctgggcacgctgctgcacgcgttcgactggagcttgcctgacggcgaggagaagctggacatgagcgagacgttcggcctcgcgctgcctaaggctgtgccgctccgcgccgtcgtcacgccacggctcgtgccggaagcctacgcctgagctagctgtcagtggttcgcgtgtgttgttgttgccttcctgcatacgtacataacttgttatgaatcctacacttgcattggttgcctagtaccagtatttgaaattatatacggaccttgcatggtttcagttttttgacacaaccgatgatggttgatcagaagacggatctgtagagttgttctttaacaatgcgctaacctatatatcaagggactgtttggttttccatgactaaaatt

SEQ ID NO: 5 - DNA, 1599 bp, synthetic construct
  misc_feature 1..1599: note = cDNA of SEQ ID NO: 4
  source 1..1599: mol_type = other DNA
atgaagctcgccgcgttgtgcaccgaccccgtcgtgctctcctgcgccttcctctgcctcctcctccacctcgctctccggtccctgctgcacccttcttctgccgcctcttcctctggccgccgcgccggcgccggcggccacctcccgccggggccaccgggcctgccgatcctcggcgcgctgccactcgtcggccctgccccgcatgccggcctggccgcgctggcgcgcaagtacggtcccatcatgtacctgaagatgggcacgacgggcgtcgtggtggcgtcgtccccgtgcgcggcgaagacgttcctcaaggcgctggacgcgaagtacgccaaccggccggcggtggcgagcgccgccgacatcacgtaccggcgccagaacatggtgttcgcggactacgggcccaagtggaagctgatgcggaagctcgccagcgtgcacctgctcggggcgcgcgcgctcgccgactgggcgtgcgtgcggcgcgacgaggccggccacctgctccgcggcgtggcggaggcggccgcggcccggcggcccgtcgtcgtgccggagctgctcgtgtgcgcgctcgccaacatcgtcgggcagatcaccgtcagcaagcgggtgttcgacgcgcagggggatgactcaaacaggtacaaggacatgatcgtgtcgctgctgacgggcacgggcatgttcaacatcagcgacttcgtgccggcgctggcgcgtctggacctgcagggcgtgcaggccaagctgcggcggatccaccaccagttcgacggcctcatcaccaagctgctggccgagcacgccgcgacggcggcggaccgcgcgcgccagggccgccaggacttcgtcgacaggctccgcgccacgatggacgccggcgccgacgacgagagcggcgagaccatcaccgaggtcaacatcaagggcctcatcttcgacatgttcacggcgggcacggacacgtcgtccatcatcgtggagtgggcgatggcggagatgctcaagaacccgtccgtgatggcgcgcgcgcaggaggagctggaccgcgtggtgggccgcggccgccgcctggaggagtcggacctgcccagcctcccctacctgcaggccgtctgcaaggaggccatgcggctgcacccgtcgacgccgctcagcctcccgcacttctccttcgacgcctgcgacgacgacgtcgccgccggcggctaccgcgtcccggccaacacccggctgctcatcaacatctgggccatcggccgggacccggcggcgtggaagaagcccctggagttccggcccgagcggttcctgccgggcggcggcgccgagaaggtcgacccgatggggaactgcttcgagctcatcccgttcggcgccggcaggaggatctgcgccgggaagctggccggcatggtgttcgtgcagtacttcctgggcacgctgctgcacgcgttcgactggagcttgcctgacggcgaggagaagctggacatgagcgagacgttcggcctcgcgctgcctaaggctgtgccgctccgcgccgtcgtcacgccacggctcgtgccggaagcctacgcctag

SEQ ID NO: 6 - protein, 532 aa, Sorghum bicolor
MKLAALCTDPVVLSCAFLCLLLHLALRSLLHPSSAASSSGRRAGAGGHLPPGPPGLPILGALPLVGPAPHAGLAALARKYGPIMYLKMGTTGVVVASSPCAAKTFLKALDAKYANRPAVASAADITYRRQNMVFADYGPKWKLMRKLASVHLLGARALADWACVRRDEAGHLLRGVAEAAAARRPVVVPELLVCALANIVGQITVSKRVFDAQGDDSNRYKDMIVSLLTGTGMFNISDFVPALARLDLQGVQAKLRRIHHQFDGLITKLLAEHAATAADRARQGRQDFVDRLRATMDAGADDESGETITEVNIKGLIFDMFTAGTDTSSIIVEWAMAEMLKNPSVMARAQEELDRVVGRGRRLEESDLPSLPYLQAVCKEAMRLHPSTPLSLPHFSFDACDDDVAAGGYRVPANTRLLINIWAIGRDPAAWKKPLEFRPERFLPGGGAEKVDPMGNCFELIPFGAGRRICAGKLAGMVFVQYFLGTLLHAFDWSLPDGEEKLDMSETFGLALPKAVPLRAVVTPRLVPEAYA

SEQ ID NO: 7 - DNA, 3752 bp, Sorghum bicolor
  source 1..3752: mol_type = unassigned DNA
atatagcttgtctgagttccgagtacgcagccgcaggagcgcagcgtgctgcagggtgcaacgcctcatcaggaatataaagccgtcgtccaggagacacccttagcttgggaaggtcctcctccctggcacagcacagtagcacgccaccatgcagctcgccgcgttgtgcatcgaccccctcgtgctctcctgcgccttcctctgcctcctcctccacgtcgcgctccgctccctgctgcacccttcttctgcctcgtcctccggccgccgcggcggccaccaccagctcccgccggggccaccgggactaccgatcctcggcgcgctgccactcgtcggcccagccccgcacgccggcctggccgcgctggcgcgcaagtacggtcccatcatgtacctgaagatgggcacgacgggcgtcgtggtggcgtcgtccccgggcgcggcgaggacgttcctcaaggcgctggacgcgaagtacgccaaccggccggcggtggcgagcgccgccgacatcacgtacgggtgccagaacatggtgttcgccaactacgggcccaagtggaagctgatgcggaagctcgccagcgtgcacctgctcggggcgcgcgcgctcgcggactgggcgcacgtgcggcgcgacgaggccggccacctgctccgcggcgtggcggaggcggccgcggcccggcggcccgtcgtcgtgccggaggtgctggtgtgcgccctcgccaatatcgtcgggcagatcaccgtgagtaagcgggtgttcgacgcacagggggacgagtccaacaggtcagaactgaaccgaaaggccttttcagtgaatagaatcgctcaccaaaatacgaatgtgaaatatgtgtgtgcaggtacaaggacatgatcgtgtcactgctgactggcgcgggactgttcaacatcagcgacttcgtgccggcgctggcgcgtctggacctgcagggcgtgcaggccaagctgcggcgtgtgcaccaccagttcgacggcctcatcaccaagctgctggccgagcacgccgcgacggccgcggaccgcgcgcgcgacggccgccaggacttcgtcgacaggctccgcgccgccattgacgccggcgctgacgacgagagcggcgagaccatcaccgaggtcaacatcaagggcctcatcttcgtaagctcgctttcctcacctcagctacatacatatatactctttccttccttccattgctttcgttgttttatcaagtaaagtaatgcattccgtcgcggtaagaatgtaagatatgcagcagctagtaccgatcatgaaagaaagtaactgagaatcttgtgctcaaaataaggcattgtttagttccgaaaaaaatttaccaaaaattttagccgttaaattaaatcttgcggcatatatatggagtattaaagatataaaaataaaaattaattacatagtttgcctgtaatttatgagacgaacctttaaacctagttagtttataattggacaatatttgtcaaataaaaataaaaatgctacagtatctgaaattccaaaattttgtcaactgaacaaggccttagatgcatttaggccatgtttggtttcaaatgttaaagtttatcgctcgacacatcgaatgtttagtacacatgcatagagtactaaatatagactatttatgaaactaaaaacacagctagagagtaatttgcgagatgaatcttttaaacctaattaatctatgattagacattaattgttaaataagatgaaaatgttacaacccaaccaagcacccccttacgtcgtcgcgttggtgcggccgagcaggagagttaggtggactagtgggttgttggaaagtgacgatcgaagcgaaagcacacattagctcgagctgatatgtttggtttatggtctaattcataccgtgagggcctgcaggttctgagtctttcatgattagtcaaggtgatatcggtagctgatggctttgtttggtaccaaagtgcagactagagcaaaatatggttcaaatgttgataacatttggtaccgtgaagtgaatagaatacctaccggttgtgccatggttgtcctcaaaattcatgtttttttaataccccatctattttaaattacaagacattttgacttttctagataaattgatttttctatgtatctagatatagtgtctatctaaatgtatagtaaagattacatatatttaaaaaagctaaacattttataattagaatagggggagtatataattaaaagagagtatctgtttatagtgatatttctcaagtacagtccacatacagaatgcaccgagaccattctaaaacctcaaacatgttgcaaagatctaatgttctggaaccaaaatatatgaactgaatatcgacgattgaatcattgggtacaacaaatattgtagatttcaggatctatgaccattgatccatcaagttggacgcttgacttctccgcagctaccaatggtgacgtcaatgacctggagttaatatctctacggagtattactcgtccttgcacaaggatgaccttatcgtggccatgaaaatagacatatagacaatgtagggtttatctagaataatcactaataagaataagatgagattgaataacatccaaacagtgtcggatctactcatttggttaattggtaactttacccacaaaacactgaagtaattcgtgtccatttaattttttccgtcctatacaatgtttgcatgcaacgcatccaggacatgttcacggcgggcacggacacatcgtcgatcatcgtggagtgggcgatgtctgaaatgctcaagaacccgcccgtcatggcacgagcgcaggaggagctggaccgcacggtcggccgcggccgccgcctggaagagtcggacctgcccagcctcccctacctgcaggccgtctgcaaggaggccatgcggctgcacccgtcgacgccgctcagcctcccgcacttctccttcgacgcctgcgacgacgtcggcggcggctaccgcgtcccggccaacacccggctgctcatcaacgtctgggccatcggccgggacccggcgacgtgggaggcgcccctcgagttccggccagagcggttcctgccgggggccgcggcggagaaggtcgacccgatggggaactacttcgagctcatccccttcggcgccggccggaggatctgcgccgggaagctggccggcatggtgttcgtgcagtacttcctgggcacgctgctgcacgcgttcaactggcgcctcgccgacggcgaggagctggacatgcgcgagacgttcgggctcacgctgcctaaggccgtaccgctccgcgccattgtcacgccgcggctcctgccggaagcctatgcttgagttagttgtcacttgtcagtctcagtccgcgctgtggaaggatgatcggaaggtttgtgcttgccaacctgcatacatattctgtttatatgtttttttatagaaactacatggtattcatcatatacacaaacgcacatctctctatgcttaattgcaaggcatttgatatggaaaatccaccttggattcaatagtttgacaactactgatcttactgaacaaatgattattccggtccattaaaatgaacttttacaggcagttttgtaagaccgttcatgtgcttcaacttcaagcagtaaggcctcatttggttgcatc

SEQ ID NO: 8 - DNA, 1584 bp, synthetic construct
  misc_feature 1..1584: note = cDNA of SEQ ID NO: 7
  source 1..1584: mol_type = other DNA
atgcagctcgccgcgttgtgcatcgaccccctcgtgctctcctgcgccttcctctgcctcctcctccacgtcgcgctccgctccctgctgcacccttcttctgcctcgtcctccggccgccgcggcggccaccaccagctcccgccggggccaccgggactaccgatcctcggcgcgctgccactcgtcggcccagccccgcacgccggcctggccgcgctggcgcgcaagtacggtcccatcatgtacctgaagatgggcacgacgggcgtcgtggtggcgtcgtccccgggcgcggcgaggacgttcctcaaggcgctggacgcgaagtacgccaaccggccggcggtggcgagcgccgccgacatcacgtacgggtgccagaacatggtgttcgccaactacgggcccaagtggaagctgatgcggaagctcgccagcgtgcacctgctcggggcgcgcgcgctcgcggactgggcgcacgtgcggcgcgacgaggccggccacctgctccgcggcgtggcggaggcggccgcggcccggcggcccgtcgtcgtgccggaggtgctggtgtgcgccctcgccaatatcgtcgggcagatcaccgtgagtaagcgggtgttcgacgcacagggggacgagtccaacaggtacaaggacatgatcgtgtcactgctgactggcgcgggactgttcaacatcagcgacttcgtgccggcgctggcgcgtctggacctgcagggcgtgcaggccaagctgcggcgtgtgcaccaccagttcgacggcctcatcaccaagctgctggccgagcacgccgcgacggccgcggaccgcgcgcgcgacggccgccaggacttcgtcgacaggctccgcgccgccattgacgccggcgctgacgacgagagcggcgagaccatcaccgaggtcaacatcaagggcctcatcttcgacatgttcacggcgggcacggacacatcgtcgatcatcgtggagtgggcgatgtctgaaatgctcaagaacccgcccgtcatggcacgagcgcaggaggagctggaccgcacggtcggccgcggccgccgcctggaagagtcggacctgcccagcctcccctacctgcaggccgtctgcaaggaggccatgcggctgcacccgtcgacgccgctcagcctcccgcacttctccttcgacgcctgcgacgacgtcggcggcggctaccgcgtcccggccaacacccggctgctcatcaacgtctgggccatcggccgggacccggcgacgtgggaggcgcccctcgagttccggccagagcggttcctgccgggggccgcggcggagaaggtcgacccgatggggaactacttcgagctcatccccttcggcgccggccggaggatctgcgccgggaagctggccggcatggtgttcgtgcagtacttcctgggcacgctgctgcacgcgttcaactggcgcctcgccgacggcgaggagctggacatgcgcgagacgttcgggctcacgctgcctaaggccgtaccgctccgcgccattgtcacgccgcggctcctgccggaagcctatgcttga

SEQ ID NO: 9 - protein, 527 aa, Sorghum bicolor
MQLAALCIDPLVLSCAFLCLLLHVALRSLLHPSSASSSGRRGGHHQLPPGPPGLPILGALPLVGPAPHAGLAALARKYGPIMYLKMGTTGVVVASSPGAARTFLKALDAKYANRPAVASAADITYGCQNMVFANYGPKWKLMRKLASVHLLGARALADWAHVRRDEAGHLLRGVAEAAAARRPVVVPEVLVCALANIVGQITVSKRVFDAQGDESNRYKDMIVSLLTGAGLFNISDFVPALARLDLQGVQAKLRRVHHQFDGLITKLLAEHAATAADRARDGRQDFVDRLRAAIDAGADDESGETITEVNIKGLIFDMFTAGTDTSSIIVEWAMSEMLKNPPVMARAQEELDRTVGRGRRLEESDLPSLPYLQAVCKEAMRLHPSTPLSLPHFSFDACDDVGGGYRVPANTRLLINVWAIGRDPATWEAPLEFRPERFLPGAAAEKVDPMGNYFELIPFGAGRRICAGKLAGMVFVQYFLGTLLHAFNWRLADGEELDMRETFGLTLPKAVPLRAIVTPRLLPEAYA

SEQ ID NO: 10 - DNA, 187 bp, Zea mays
  source 1..187: mol_type = unassigned DNA
cttctgcccagaagcgggcccagacatttgagattgggtattcaaaaattcaaaagattaaagaatttagtgttctaacgctattttatgcaatacattattgacaaattagtgttctaacactatagatcaccaaaaacatgggtattcaatgaatacccatgaaacccccctgggcccgcccatg

SEQ ID NO: 11 - DNA, 4402 bp, Zea mays
  source 1..4402: mol_type = unassigned DNA
atgcagctcgcggcgttgtgcaccgaccccgtggtgctgtgcagcgccttcctctgcctcctcctccacgtggctctccgctcgctgctgcaccctccttctgcccagaagcgggcccagacatttgagattgggtattcaaaaattcaaaagattaaagaatttagtgttctaacgctattttatgcaatacattattgacaaattagtgttctaacactatagatcaccaaaaacatgggtattcaatgaatacccatgaaacccccctgggcccgcccatgcttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 12 - DNA, 387 bp, Zea mays
  misc_feature 101..287: note = insertion of 187 nucleotides as present in QTL line
  source 1..387: mol_type = unassigned DNA
accatgcagctcgcggcgttgtgcaccgaccccgtggtgctgtgcagcgccttcctctgcctcctcctccacgtggctctccgctcgctgctgcaccctccttctgcccagaagcgggcccagacatttgagattgggtattcaaaaattcaaaagattaaagaatttagtgttctaacgctattttatgcaatacattattgacaaattagtgttctaacactatagatcaccaaaaacatgggtattcaatgaatacccatgaaacccccctgggcccgcccatgcttctgccgcctcttcctccgggcgccgcgggcagctcccgccggggccaccgggcctgccgatcctcggcgcgctgccactcgtgggcccagccccgca

SEQ ID NO: 13 - DNA, 201 bp, Zea mays
  misc_feature 101: note = single nucleotide polymorphism
  source 1..201: mol_type = unassigned DNA
tgatgaaagccgtgcaggagtaaatagraaamtggagttgtggcgtgaractctagaatccaaacgttttagactctttagaactaaaaccgaatatatawgttgtgactttggcactactacatatgacgaaggagatattagtttggaaggtcaagtagtgccctgraaggataccttttggtatttagtatcaatgct

SEQ ID NO: 14 - DNA, 121 bp, Zea mays
  misc_difference 58: note = n is a, c, g, or t
  misc_feature 61: note = single nucleotide polymorphism
  source 1..121: mol_type = unassigned DNA
caaggaacctaggatgggggcggattcattaacaaggaggggggccctaatcccctgncgrtctgcacgggcaataatctagtcagaccccgaggtcagtagaggcggaggacgcgtcacg

SEQ ID NO: 15 - DNA, 121 bp, Zea mays
  misc_feature 61: note = single nucleotide polymorphism
  source 1..121: mol_type = unassigned DNA
cagcacatattgtgttgaaatgaatgagcacactgcatccatatcaccactggtacacaarggcatagtttaagtcatgttatgatgtagcgcaccgagcaaggtattttgacacacataa

SEQ ID NO: 16 - DNA, 101 bp, Zea mays
  misc_feature 51: note = single nucleotide polymorphism
  source 1..101: mol_type = unassigned DNA
aatgtgggaagagctggagcagcaaatctagacgtcattcaagaagtcgartatgtaaaggaagatgctagaattatatacctccttgaatgcctccaaaa

SEQ ID NO: 17 - DNA, 101 bp, Zea mays
  misc_feature 51: note = single nucleotide polymorphism
  source 1..101: mol_type = unassigned DNA
ctctttctgcagctcgtgatcgagttgtgtgtgctccattcccaaattatrtatgatcgattagtagcgttaggggttaaccagtagccgctgtgtacttg

SEQ ID NO: 18 - DNA, 201 bp, Zea mays
  misc_feature 101: note = single nucleotide polymorphism
  source 1..201: mol_type = unassigned DNA
ttatgggggaggcagattgtcgtggggcgcgggggagggcgagcttgggggcgccgcagagcgagggacattgccgagattgccatggagtgagggcacgrcgagattgccgcgagggagagggcgcttgccgaggcagattgccgcgggggaggggcgcttgccgaggcggagacgtggtgaagccgtggtggacgccca

SEQ ID NO: 19 - DNA, 4215 bp, synthetic construct
  misc_feature 1..4215: note = genomic DNA of F35H mutant P434L
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgcctcgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 20 - protein, 527 aa, synthetic construct
  REGION 1..527: note = amino acid sequence of F35H mutant P434L
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWERPLDFRLERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 21 - DNA, 4215 bp, synthetic construct
  misc_feature 1..4215: note = genomic DNA of F35H mutant W426stop
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctaggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 22 - protein, 425 aa, synthetic construct
  REGION 1..425: note = amino acid sequence of F35H mutant W426stop
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEA

SEQ ID NO: 23 - DNA, 4215 bp, synthetic construct
  misc_feature 1..4215: note = genomic DNA of F35H mutant R252W
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgtggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 24 - protein, 527 aa, synthetic construct
  REGION 1..527: note = amino acid sequence of F35H mutant R252W
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLWRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWERPLDFRPERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 25 - DNA, 4215 bp, synthetic construct
  misc_feature 1..4215: note = genomic DNA of F35H mutant R405H
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccacgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 26 - protein, 527 aa, synthetic construct
  REGION 1..527: note = amino acid sequence of F35H mutant R405H
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYHVPANTRLLVNVWAIGRDPEAWERPLDFRPERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 27 - DNA, 4215 bp, synthetic construct
  misc_feature 1..4215: note = genomic DNA of F35H mutant P407L
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcctggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 28 - protein, 527 aa, synthetic construct
  REGION 1..527: note = amino acid sequence of F35H mutant P407L
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVLANTRLLVNVWAIGRDPEAWERPLDFRPERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 29 - DNA, 4215 bp, synthetic construct
  misc_feature 1..4215: note = genomic DNA of F35H mutant E427K
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctggaagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 30 - protein, 527 aa, synthetic construct
  REGION 1..527: note = amino acid sequence of F35H mutant E427K
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWKRPLDFRPERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 31 - DNA, 4215 bp, synthetic construct
  misc_feature 1..4215: note = genomic DNA of F35H mutant G450R
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctgaggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 32 - protein, 527 aa, synthetic construct
  REGION 1..527: note = amino acid sequence of F35H mutant G450R
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWERPLDFRPERFLPGGGAEKVDPLRNCFELIPFGAGRRICAGKLAGMVFVQYFLGTLLHAFDWRLPDGEEKLDMSETFGLALPKAVPLRAVATPRLVPEAYA

SEQ ID NO: 33 - DNA, 4215 bp, synthetic construct
  misc_feature 1..4215: note = genomic DNA of F35H mutant P429S
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggtccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 34 - protein, 527 aa, synthetic construct
  REGION 1..527: note = amino acid sequence of F35H mutant P429S
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWERSLDFRPERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 35 - DNA, 4215 bp, synthetic construct
  misc_feature 1..4215: note = genomic DNA of F35H mutant P429L
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggctcctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 36 - protein, 527 aa, synthetic construct
  REGION 1..527: note = amino acid sequence of F35H mutant P429L
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWERLLDFRPERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 37 - DNA, 4215 bp, synthetic construct
  misc_feature 1..4215: note = genomic DNA of F35H mutant R436C
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagtgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 38 - protein, 527 aa, synthetic construct
  REGION 1..527: note = amino acid sequence of F35H mutant R436C
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWERPLDFRPECFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 39 - DNA, 4215 bp, synthetic construct
  misc_feature 1..4215: note = genomic DNA of F35H mutant Q44stop
  source 1..4215: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggtagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 40 - protein, 43 aa, synthetic construct
  REGION 1..43: note = amino acid sequence of F35H mutant Q44stop
MQLAALCTDPVVLCSAFLCLLLHVALRSLLHPPSAASSSGRRG